(19) United States
(12) Patent Application Publication
(10) Pub. No.: US 2015/0104441 A1
(43) Pub. Date: Apr. 16, 2015

(54) IDENTIFICATION OF DISEASE-DRIVING ANTIGENS

(71) Applicant: OSLO UNIVERSITETSSYKEHUS HF, OSLO (NO)

(72) Inventors: Johanna Olweus, Eiksmarka (NO); Trygve Holmøy, Oslo (NO); Andreas Lossius, Oslo (NO)

(21) Appl. No.: 14/512,737

(22) Filed: Oct. 13, 2014

Related U.S. Application Data
(60) Provisional application No. 61/889,782, filed on Oct. 11, 2013.

(51) Int. Cl. G01N 33/50; C12Q 1/68

(52) U.S. Cl. CPC G01N 33/5023; C12Q 1/6883; C12Q 2600/118; C12Q 2600/156; C12Q 2600/16; G01N 2800/285; G01N 2800/28; C12Q 2600/136
USPC 424/130.1; 506/2; 514/44 R

(57) ABSTRACT

Provided herein is technology relating to treating disease and particularly, but not exclusively, to methods for identifying disease-related antigens by assessing T cell receptor gene frequencies in diseased subjects.

### CROSS-REFERENCE TO RELATED APPLICATIONS

The present application claims priority to pending U.S. Provisional Patent Application No. 61/889,782, filed Oct. 11, 2013, the contents of which are incorporated by reference in their entirety.

### FIELD OF INVENTION

Provided herein is technology relating to diagnosing disease and particularly, but not exclusively, to methods for identifying disease-related antigens by assessing T cell receptor gene frequencies in diseased subjects.

### BACKGROUND

Infectious, inflammatory, and autoimmune diseases involve immunological attack of cells by an organism's immune system. While a response to infection directs an immunological attack against heterologous (e.g., “non-self”) cells, auto-immune and inflammatory disorders provoke immunological attack against an organism's own (“self”) cells and tissues. One approach toward gaining insight into preventing and treating auto-immune and inflammatory disorders involves modulating (e.g., targeting, masking, etc.) the antigens associated with the aberrant anti-self immune response in these disorders. To date, however, the specific antigens driving anti-self immune responses are not known for most inflammatory and autoimmune diseases, thus impeding diagnosis, drug development, prophylaxis, and treatment. As such, identifying the antigens provoking the adaptive cellular immune response into an “anti-self” attack would provide for the development of specific treatments to ameliorate such diseases. Consequently, methods are needed for identifying disease-related antigens in diseases such as autoimmune and inflammatory diseases.

### SUMMARY

Provided herein is technology relating to diagnosing disease and particularly, but not exclusively, to methods for identifying disease-related antigens by assessing T cell receptor gene frequencies in diseased subjects. The technology provides a novel method to screen antigens involved in disease with high sensitivity. In some embodiments, the technology provides for testing immune responses against a number of antigens using peripheral blood samples drawn from a subject and/or cells harvested from a disease-affected organ or tissue from the subject.

For example, some embodiments of the technology provide a method for identifying disease-related antigens by assessing increases in T cell receptor (TCR) gene frequencies by, e.g., collecting a peripheral blood sample and a disease-related sample (e.g., cells from an organ and/or tissue affected by disease) from an individual (e.g., subject, patient) having or suspected of having an autoimmune and/or inflammatory disease; stimulating the blood sample with candidate and control antigens in vitro; isolating T cells responding to each antigen to provide antigen responding samples; sequencing TCR genes from the unmanipulated blood sample and from the antigen responding samples and from the disease-related sample; and comparing the relative frequencies of TCR genes in the sequenced samples. A disease-related antigen is identified by high frequencies of the same TCR genes in the disease-related sample and one or more antigen-responding samples.

Accordingly, provided herein are embodiments of methods for identifying a disease-related antigen, the methods comprising sequencing a set of T cell receptor genes from a disease-related sample from a subject having a disease; sequencing a set of T cell receptor genes from a control sample; comparing the T cell receptor gene frequencies of the set of T cell receptor genes from the disease-related sample with the T cell receptor gene frequencies of the set of T cell receptor genes from the control sample to identify T cell receptor genes enriched in the disease-related sample, wherein the T cell receptor genes enriched in the disease-related sample identify a disease-related antigen associated with the disease. In this way, the specificity of very low numbers of T cells present in the disease-related sample can be identified, by creating reference libraries from T cell responses to memory antigens in peripheral blood, which is easily available and contains large frequencies of memory T cells. In some embodiments, the disease is an autoimmune or inflammatory disease. In some embodiments, the T cell genes encode T cell receptor alpha- and/or beta-chain polypeptides. In some embodiments, the T cell genes encode complementarity determining regions 3 (CDR3). In preferred embodiments, the disease-related sample is tissue from the central nervous system or cerebrospinal fluid and the control sample is a blood sample from the same subject. In some embodiments, additional control samples are included from a different subject.

Moreover, some embodiments provide a method of identifying a disease-related antigen, the method comprising contacting a peripheral blood sample from a subject diagnosed with an inflammatory or an autoimmune disease with a test antigen and a control antigen; isolating T cells from the sample that respond to said test antigen to provide test-antigen responsive T cells and isolating T cells from the sample that respond to said control antigen to provide control-antigen responsive T cells; sequencing T-cell receptor genes from said test-antigen responsive T cells to provide test-antigen responsive genes and sequencing T-cell receptor genes from said control-antigen responsive T cells to provide control-antigen responsive genes; and comparing the frequency of test-antigen responsive gene sequences and the frequency of control-antigen responsive gene sequences with the frequencies of the corresponding T-cell receptor genes in a disease-related sample from the same individual, wherein the test-antigen responsive gene sequences enriched in the disease-related sample identify a disease-related antigen associated with the disease. In some embodiments the T cells are CD4+ or CD8+ T cells. In some embodiments the disease is an autoimmune or inflammatory disease. In some embodiments the T cell receptor genes encode T cell receptor alpha- and/or beta-chain polypeptides. In some embodiments the T cell receptor genes encode complementarity determining regions 3 (CDR3). In some embodiments the disease-related sample is tissue from the central nervous system or cerebrospinal fluid. In some embodiments, the methods further comprise contacting a control sample from a subject not diagnosed with an inflammatory or an autoimmune disease with the test antigen and the control antigen.

In related embodiments is provided a method of treating a disease comprising an aberrant immunological response to a disease-related antigen, the method comprising identifying the disease-related antigen according to a method described herein and minimizing the immunological response to the disease-related antigen in a patient. In related embodiments is provided a composition comprising an antigenic peptide identified according to a method described herein.

Such methods and compositions find use in treating and/or diagnosing disease. For example, some embodiments relate to the use of a disease-related antigen identified according to a method provided herein as a biomarker for a disease. Some embodiments relate to the use of disease-related antigen identified according to a method provided herein in the preparation of a medicament, e.g., for the treatment and/or prevention of disease.

Additional embodiments will be apparent to persons skilled in the relevant art based on the teachings contained herein.

### BRIEF DESCRIPTION OF THE DRAWINGS

These and other features, aspects, and advantages of the present technology will become better understood with regard to the following drawings:

FIG. 1 is a series of plots showing the clonal distribution of T cells in cerebrospinal fluid (CSF) and blood of 10 patients with MS and 11 controls with other neuroinflammatory diseases (OND). FIG. 1 shows the frequency distributions of CDR3 sequences from CSF and blood of patients with MS and OND, and from CSF of the latter group divided into acute (disease duration<2 months; n=6) and chronic (disease duration>2 months; n=5) inflammation.

FIG. 2 is a plot showing V-gene usage of T cells in the cerebrospinal fluid (CSF) and blood from patients with MS and controls with other neuroinflammatory diseases (OND).

FIG. 3 is a plot showing J-gene usage of T cells in the cerebrospinal fluid and blood from patients with MS and controls with other neuroinflammatory diseases.

FIG. 4 is a series of plots showing clonal distribution of T cells in cerebrospinal fluid (CSF) and blood of 10 patients with MS and 11 controls with other neuroinflammatory diseases (OND).

FIG. 5 is a series of diagrams and plots showing the overlap of TCRβ CDR3 sequences between paired cerebrospinal fluid (CSF) and blood samples from 10 patients with MS and 11 controls with other neuroinflammatory diseases (OND). FIG. 5A shows Venn diagrams proportioned to the overlap between CDR3 sequences from CSF and blood. FIG. 5B shows the degree of overlap between CDR3 sequences from CSF and blood according to their frequency. FIG. 5C shows Bland-Altman plots of CDR3 sequences expanded above 0.1% in serial blood draws from 3 MS patients (n=189), and in CSF and blood of 10 patients with MS (n=932) and 11 with OND (n=903). FIG. 5D shows the sharing of expanded (>0.1%) CDR3 sequences.

FIG. 6 is a series of plots showing the frequencies of CDR3 sequences expanded above 0.1% in cerebrospinal fluid (CSF) (FIG. 6A) and blood (FIG. 6B) in MS-4 and MS-5 at baseline (t=0) and 14 months later. Each data point represents a distinct TCRβ CDR3 sequence.

FIG. 7 is a series of plots showing the frequencies of EBV- and Influenza A-reactive T cells in cerebrospinal fluid (CSF) and blood of patients with MS (n=10) and controls with other neuroinflammatory diseases (OND; n=11). FIG. 7A shows the strategy used to sort virus-reactive CD4+ and CD8+ T cells after stimulating twice with the antigen. FIG. 7B shows the estimated total proportion of CD8+ EBV- and Influenza A-reactive T cells in blood and CSF of patients with MS and controls with OND; Wilcoxon signed rank test; *P<0.025; **P<0.005. FIG. 7C shows the estimated total proportion of CD4+ EBV- and Influenza A-reactive T cells in blood and CSF of patients with MS and controls with OND; Wilcoxon signed rank test; *P<0.025; **P<0.005. FIG. 7D shows the proportion of EBV-reactive CDR3 sequences among the 50 most expanded clones in the CSF and blood of patients with MS and controls with OND.

FIG. 8 is a plot showing estimated proportions of Varizella zoster virus (VZV), Epstein-Barr virus (EBV), and Influenza A-reactive CD4+ and CD8+ T cells in the cerebrospinal fluid (CSF) and blood of a patient with VZV encephalitis. Results are given as percentage of whole.

FIG. 9 is a plot showing the frequencies of TCRβ CDR3 sequences associated with public CD8+ T cell responses against EBV and cytomegalovirus (CMV) in the CSF and blood of 10 MS patients and 11 controls with other neuroinflammatory diseases (OND).

It is to be understood that the figures are not necessarily drawn to scale, nor are the objects in the figures necessarily drawn to scale in relationship to one another. The figures are depictions that are intended to bring clarity and understanding to various embodiments of apparatuses, systems, and methods disclosed herein. Wherever possible, the same reference numbers will be used throughout the drawings to refer to the same or like parts. Moreover, it should be appreciated that the drawings are not intended to limit the scope of the present teachings in any way.

### DETAILED DESCRIPTION

Provided herein is technology relating to treating disease and particularly, but not exclusively, to methods for identifying disease-related antigens by assessing T cell receptor gene frequencies in diseased subjects. The technology provides a strategy to chart antigen-reactive TCR repertoires on a molecular level for diseases involving an immune response.

### DEFINITIONS

To facilitate an understanding of the present technology, a number of terms and phrases are defined below. Additional definitions are set forth throughout the detailed description.

Throughout the specification and claims, the following terms take the meanings explicitly associated herein, unless the context clearly dictates otherwise. The phrase “in one embodiment” as used herein does not necessarily refer to the same embodiment, though it may. Furthermore, the phrase “in another embodiment” as used herein does not necessarily refer to a different embodiment, although it may. Thus, as described below, various embodiments of the invention may be readily combined, without departing from the scope or spirit of the invention.

In addition, as used herein, the term “or” is an inclusive “or” operator and is equivalent to the term “and/or” unless the context clearly dictates otherwise. The term “based on” is not exclusive and allows for being based on additional factors not described, unless the context clearly dictates otherwise. In addition, throughout the specification, the meaning of “a”, “an”, and “the” include plural references. The meaning of “in” includes “in” and “on.”

As used herein, the term “pharmaceutical composition” refers to the combination of an active agent with a carrier, inert or active, making the composition especially suitable for therapeutic use.

The terms “pharmaceutically acceptable” or “pharmacologically acceptable”, as used herein, refer to compositions that do not substantially produce adverse reactions, e.g., toxic, allergic, or immunological reactions, when administered to a subject.

As used herein, the term “antibody” is used in its broadest sense to refer to whole antibodies, monoclonal antibodies (including human, humanized, or chimeric antibodies), polyclonal antibodies, and antibody fragments that can bind antigen (e.g., Fab′, F′(ab)₂, Fv, single chain antibodies), comprising complementarity determining regions (CDRs) of the foregoing as long as they exhibit the desired biological activity.

As used herein, “antibody fragments” comprise a portion of an intact antibody, preferably the antigen binding or variable region of the intact antibody. Examples of antibody fragments include Fab, Fab′, F(ab)₂, and Fv fragments; diabodies; linear antibodies (Zapata et al., Protein Eng. 8(10): 1057-1062 (1995)); single-chain antibody molecules; and multispecific antibodies formed from antibody fragments.

A molecule that “specifically binds to” or is “specific for” another molecule is one that binds to that particular molecule without substantially binding to any other molecule.

As used herein the term, “in vitro” refers to an artificial environment and to processes or reactions that occur within an artificial environment. In vitro environments may include, but are not limited to, test tubes and cell cultures. The term “in vivo” refers to the natural environment (e.g., an animal or a cell) and to processes or reactions that occur within a natural environment.

As used herein, the term “administration” refers to the act of giving a drug, prodrug, antibody, vaccine, or other agent, or therapeutic treatment to a physiological system (e.g., a subject or in vivo, in vitro, or ex vivo cells, tissues, and organs). Exemplary routes of administration to the human body can be through the eyes (ophthalmic), mouth (oral), skin (transdermal), nose (nasal), lungs (inhalant), oral mucosa (buccal), ear, by injection (e.g., intravenously, subcutaneously, intratumorally, intraperitoneally, etc.) and the like.

“Coadministration” refers to administration of more than one chemical agent or therapeutic treatment to a physiological system (e.g., a subject or in vivo, in vitro, or ex vivo cells, tissues, and organs). As used herein, administration “in combination with” one or more further therapeutic agents includes simultaneous (concurrent) and consecutive administration in any order. “Coadministration” of therapeutic treatments may be concurrent, or in any temporal order or physical combination.

As used herein, “carriers” include pharmaceutically acceptable carriers, excipients, or stabilizers which are nontoxic to the cell or mammal being exposed thereto at the dosages and concentrations employed. Often the physiologically acceptable carrier is an aqueous pH-buffered solution. Examples of physiologically acceptable carriers include buffers such as phosphate, citrate, and other organic acids; antioxidants including ascorbic acid; low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, arginine, or lysine; monosaccharides, disaccharides, and other carbohydrates including glucose, mannose, or dextrins; chelating agents such as EDTA; sugar alcohols such as mannitol or sorbitol; salt-forming counterions such as sodium; and/or nonionic surfactants.

As used herein, the terms “protein,” “polypeptide,” and “peptide” refer to a molecule comprising amino acids joined via peptide bonds. In general, “peptide” is used to refer to a sequence of 20 or less amino acids and “polypeptide” is used to refer to a sequence of greater than 20 amino acids.

As used herein, the term, “synthetic polypeptide,” “synthetic peptide”, and “synthetic protein” refer to peptides, polypeptides, and proteins that are produced by a recombinant process (i.e., expression of exogenous nucleic acid encoding the peptide, polypeptide, or protein in an organism, host cell, or cell-free system) or by chemical synthesis.

As used herein, the term “protein of interest” refers to a protein encoded by a nucleic acid of interest.

As used herein, the term “native” (or wild type) when used in reference to a protein refers to proteins encoded by the genome of a cell, tissue, or organism, other than one manipulated to produce synthetic proteins.

As used herein, “domain” (typically a sequence of three or more, generally 5 or 7 or more amino acids) refers to a portion of a molecule, such as proteins or the encoding nucleic acids, that is structurally and/or functionally distinct from other portions of the molecule and is identifiable. For example, domains include those portions of a polypeptide chain that can form an independently folded structure within a protein made up of one or more structural motifs and/or that is recognized by virtue of a functional activity, such as proteolytic activity. As such, a domain refers to a folded protein structure that retains its tertiary structure independently of the rest of the protein. Generally, domains are responsible for discrete functional properties of proteins, and in many cases may be added, removed or transferred to other proteins without loss of function of the remainder of the protein and/or of the domain.

A protein can have one, or more than one, distinct domains. For example, a domain can be identified, defined or distinguished by homology of the sequence therein to related family members, such as homology to motifs that define a protease domain or a gla domain. In another example, a domain can be distinguished by its function, such as by proteolytic activity, or an ability to interact with a biomolecule, such as DNA binding, ligand binding, and dimerization. A domain independently can exhibit a biological function or activity such that the domain independently or fused to another molecule can perform an activity, such as, for example proteolytic activity or ligand binding. A domain can be a linear sequence of amino acids or a non-linear sequence of amino acids. Many polypeptides contain a plurality of domains. Some domains are known and can be identified by those of skill in the art. It is to be understood that it is well within the skill in the art to recognize particular domains by name. If needed, appropriate software can be employed to identify domains.

As used herein, the term “host cell” refers to any eukaryotic cell (e.g., mammalian cells, avian cells, amphibian cells, plant cells, fish cells, insect cells, yeast cells), and bacteria cells, and the like, whether located in vitro or in vivo (e.g., in a transgenic organism). The term “host cell” refers to any cell capable of replicating and/or transcribing and/or translating a heterologous gene. Thus, a “host cell” refers to any eukaryotic or prokaryotic cell, whether located in vitro or in vivo. For example, host cells may be located in a transgenic animal.

As used herein, the term “cell culture” refers to any in vitro culture of cells. Included within this term are continuous cell lines (e.g., with an immortal phenotype), primary cell cultures, finite cell lines (e.g., non-transformed cells), and any other cell population maintained in vitro, including oocytes and embryos.

The term “isolated” when used in relation to a nucleic acid or polypeptide or protein refers to a nucleic acid or polypeptide or protein sequence that is identified and separated from at least one contaminant nucleic acid or polypeptide or protein with which it is ordinarily associated in its natural source. Isolated nucleic acids or polypeptides or proteins are molecules present in a form or setting that is different from that in which they are found in nature. In contrast, non-isolated nucleic acids or polypeptides or proteins are found in the state in which they exist in nature.

The term “antigen” refers to a molecule (e.g., a protein, glycoprotein, lipoprotein, lipid, nucleic acid, or other substance) that is reactive with an antibody specific for a portion of the molecule.

The term “antigenic determinant” refers to that portion of an antigen that makes contact with a particular antibody (e.g., an epitope). When a protein or fragment of a protein is used to immunize a host animal, numerous regions of the protein may induce the production of antibodies that bind specifically to a given region or three-dimensional structure on the protein; these regions or structures are referred to as antigenic determinants. An antigenic determinant may compete with the intact antigen (e.g., the “immunogen” used to elicit the immune response) for binding to an antibody.

The terms “protein” and “polypeptide” refer to compounds comprising amino acids joined via peptide bonds and are used interchangeably. A “protein” or “polypeptide” encoded by a gene is not limited to the amino acid sequence encoded by the gene, but includes post-translational modifications of the protein.

Where the term “amino acid sequence” is recited herein to refer to an amino acid sequence of a protein molecule, “amino acid sequence” and like terms, such as “polypeptide” or “protein” are not meant to limit the amino acid sequence to the complete, native amino acid sequence associated with the recited protein molecule. Furthermore, an “amino acid sequence” can be deduced from the nucleic acid sequence encoding the protein.

The term “portion” when used in reference to a protein (as in “a portion of a given protein”) refers to fragments of that protein. The fragments may range in size from four amino acid residues to the entire amino sequence minus one amino acid (for example, the range in size includes 4, 5, 6, 7, 8, 9, 10, or 11 . . . amino acids up to the entire amino acid sequence minus one amino acid).

### DESCRIPTION OF EXEMPLARY EMBODIMENTS

Provided herein is technology relating to diagnosing disease and particularly, but not exclusively, to methods for identifying disease-related antigens by assessing T cell receptor gene frequencies in diseased subjects.

For example, some embodiments provide a method to identify antigens involved in an autoimmune disease by exposing T cells from patients having the autoimmune disease to a set of antigens and relating the particular antigen or antigens that induced enrichment of the patient T cells, to a specific repertoire of TCRs. In particular, experiments identified antigens involved in the auto-immune disorders that are manifest as multiple sclerosis. T cells are thought to play an essential role in the pathogenesis of multiple sclerosis (MS) and to be involved in the initiation and maintenance of the autoimmune response to CNS characteristic of the disease (Nylander et al., J Clin Invest. 2012; 122(4):1180-1188; McFarland et al., Nat. Immunol. 2007; 8(9):913-919). The T cells access the CNS via the cerebrospinal fluid (CSF) or cross the blood-brain barrier through the perivascular spaces, which communicate with the CSF compartment (Wilson J Clin Invest. 2010; 120(5):1368-1379). The CSF is also contiguous with the extracellular fluid of the brain, and is believed to reflect inflammation within the CNS better than blood (Stangel et al., Nat Rev Neurol. 2013; 9(5):267-276). Although moderately increased in MS (Reiber et al., Mult Scler. 1998; 4(3):111-117; Cepok S, et al. Brain. 2001; 124(pt 11):2169-2176), the low numbers of T cells in CSF constitute a limitation for characterization of these cells. Thus, the clonal composition of intrathecal T cells and their relation to T cells in blood is poorly understood. Previously, available technology allowed mapping of only a narrow part of the intrathecal T cell receptor (TCR) repertoire by cloning and sequencing individual receptors (Hafler et al., J Exp Med. 1988; 167(4):1313-1322), spectratyping (Matsumoto Y, et al. J. Immunol. 2003; 170(9):4846-4853; Muraro et al., JNeuroimmunol. 2006; 171(1-2):177-183), or flow cytometric staining for TCR variable (V) β-chain families (Jacobsen M, et al. Brain. 2002; 125(pt 3):538-550). These studies have yielded partly conflicting results with respect to clonal diversity and TCRVβ-chain usage, and the overlap between the T cell populations in CSF and blood has not been quantified.

Whereas the HLA class II association points to a role for CD4⁺ T cells in MS, clonal expansions of CD8⁺ T cells predominate in the inflammatory CNS lesions (Nylander et al, supra; Booss et al., J Neurol Sci. 1983; 62(1-3):219-232; Babbe et al., J Exp Med. 2000; 192(3):393-404). In concert with this, quantitative histological analysis of MS lesions has demonstrated a positive correlation between the numbers of infiltrating CD8⁺ T cells and acute axonal damage (Bitsch et al., Brain. 2000; 123 (Pt 6):1174-1183; Kuhlmann et al., Brain. 2002; 125(Pt 10):2202-2212). The targets of such CNS-infiltrating CD8⁺ T cells are, however, unknown.

Epstein-Barr virus (EBV) has consistently been associated with MS in epidemiological studies (Ascherio et al., Clin Exp Immunol. 2010; 160(1):120-124). Moreover, a number of observations indicate that MS patients have a perturbed immune response against EBV in blood: i) An increased tendency to spontaneous transformation of B cells (Fraser et al., Lancet. 1979; 2(8145):175-176), ii) elevated IgG levels against particular EBV nuclear antigen (EBNA)-1 epitopes (Sundstrom et al., J Neuroimmunol. 2009; 215(1-2):102-107; Sundqvist et al., Genes Immun. 2012; 13(1):14-20), iii) increased frequency, broadened specificity and frequent cross-reactivity of EBNA-1-specific CD4⁺ T cells (Lunemann J D, et al. Brain. 2006; 129(pt 6):1493-1506; Lunemann J D, et al. J Exp Med. 2008; 205(8):1763-1773), and iv) an increased but dysregulated EBV-specific CD8⁺ T cell response (Jilek S, et al. Brain. 2008; 131(pt 7):1712-1721; Jilek S, et al. J. Immunol. 2012; 188(9):4671-4680). If EBV-specific T cells play a pathogenic role in MS, they most likely access the intrathecal compartment. In support of this, CD4⁺ T cells recognizing EBV-lymphoblastoid cell lines (EBV-LCL) and EBNA-1 can be cloned from the CSF of MS patients (Holmoy et al., J Neurovirol. 2004; 10(1):52-56; Lossius A, J Neuroimmunol. 2011; 240-241: 87-96). Moreover, a recent study demonstrated an increased EBV-specific cytotoxic activity of CD8⁺ T cells in the CSF of patients with MS, but not other inflammatory neurological diseases (OIND) (Jaquiery E, et al. Eur J Immunol. 2010; 40(3):878-887). The clonal distribution and relative frequency of such T cells are, however, not known.

During the development of embodiments of the present technology, experiments were conducted to quantify EBV-specific T cells in the CSF of MS patients and to which degree they are clonally expanded, and to identify potential enrichment of EBV-specific T cells in CSF relative to blood from the same patient as compared with patients with other neuroinflammatory diseases (OND). The experiments evaluated the diversity, selectivity, and EBV-reactivity of intrathecal TCR repertoires in patients with MS and OND. Sequencing (Shendure J, Nat Biotechnol. 2008; 26(10):1135-1145) and PCR amplification were used to characterize T cell receptor (TCR)β complementarity determining region (CDR)3 genes. This approach provided a comprehensive characterization of TCR repertoires (Robins H S, et al. Blood. 2009; 114(19):4099-4107), and the relative contribution of each clone to the total repertoire can be estimated with great accuracy (Robins H, et al. J Immunol Methods. 2012; 375(1-2):14-19). The results of the experiments indicated that the TCR repertoires in the CSF of patients suffering from MS, as well as OND, were clonally diverse without any preferential V- and J-gene usage. There was little overlap detected between expanded T cell clones in CSF and blood and individual expanded CSF clones in MS were persistent over time, indicating a compartmentalized and focused intrathecal T cell response. Finally, using the novel approach to chart antigen-reactive TCRs, described above, a significant enrichment of EBV-reactive CD8+TCRs was observed, including public EBV-specific TCRs with known peptide-specificity, exclusively in the CSF of MS patients.

In particular, experiments were performed in which high-throughput sequencing of TCRβ CDR3 genes in CSF and blood of patients with MS and controls with OND provided data demonstrating the clonal composition and EBV-reactivity of the TCR repertoires. These data indicate that:
i) TCR repertoires in blood and CSF of individuals with MS and OND are clonally diverse and use an equally wide range of V- and J-genes;
ii) there is a differential expansion of T cell clones in CSF and blood of MS patients, indicating a compartmentalized expansion of clones within the CNS;
iii) these clones are persistently expanded in the CSF for at least 14 months; and
iv) CD8+ EBV-reactive T cell clones accumulate intrathecally exclusively in MS patients (not in OND).

The CSF is believed to represent a transitional station in the trafficking of antigen-experienced T cells between the blood and CNS (Wilson et al, supra), and the recruitment of T cells from the blood into the CSF is enhanced during neuroinflammation (Giunti D, et al. J Leukoc Biol. 2003; 73(5):584-590). In support of this, one study found that T cell clones isolated from MS lesions were expanded in both compartments, indicating a migration of expanded clones across the blood-brain barrier (Skulina C, et al. Proc Natl Acad Sci USA. 2004; 101(8):2428-2433). The CSF also drains interstitial fluid from the brain (Weller R O, Brain Pathol. 1996; 6(3):275-288), and may thereby reflect ongoing pathological processes within the CNS. Accordingly, a recent study demonstrated that related B cell clones populated the CNS and the CSF in MS (Obermeier B, et al. J Neuroimmunol. 2011; 233(1-2):245-248). Experiments described herein demonstrated the surprising result that TCR repertoires in CSF of MS patients were equally diverse as those in blood. Thus, the most expanded T cell clones in both compartments constituted a relatively low proportion of the total TCR repertoire. This phenomenon was not specific for MS or for chronic neuroinflammation, as OND patients with acute CNS inflammation showed the same clonal distribution. The similarities in overall distribution between the two compartments were in agreement with the lack of biased CSF Vβ- and Jβ-repertoires. The latter finding is in line with some previous studies on the intrathecal Vβ-chain usage (Jacobsen M, et al. Brain. 2002; 125(pt 3):538-550; Heard et al., J Neurol Neurosurg Psychiatry. 1999; 67(5):585-590; Gestri D, et al. J Neurol Neurosurg Psychiatry. 2001; 70(6):767-772).

Although the overall degree of diversity was similar in CSF and blood, differences were clearly revealed as to which clones were expanded in the two compartments. In fact, the more expanded in either compartment, the less likely a T cell clone was to be expanded to a similar degree in the other. Thus, little overlap was found for sequences constituting>0.1% of total TCR sequences in CSF or blood in MS, in contrast to the expected high degree of overlap if the TCR repertoire in CSF merely reflected that in blood. It is tempting to speculate that the highly diverse repertoires of low-frequent TCRs seen in CSF might represent a bystander influx of T cells, whereas TCRs that are selectively expanded in the CSF are more likely to reflect the pathogenic process. In support of the latter idea, the great majority of TCR sequences present at frequencies above 0.1% in the CSF persisted over at least 14 months in the two patients studied. This is in line with the persistence of intrathecal B cell responses, shown by the constancy of oligoclonal IgG bands over several years in the CSF of MS patients (Olsson et al., Arch Neurol. 1973; 28(6):392-399).

Here, EBV-reactive TCR sequences in CSF were quantitated by use of reference libraries established from EBV-responsive T cells sorted from peripheral blood. The reliability of this approach was underscored by i) the finding of EBV-associated public TCRβ CDR3 sequences in the EBV reference library, ii) a corresponding enrichment of influenza A-associated public TCRβ CDR3 sequences in the influenza A reference library, and iii) the high frequencies and intrathecal accumulation of VZV-reactive T cells in a patient with VZV-encephalitis. This strategy finds use in interrogation of the CSF repertoire for presence of TCR sequences specific for any antigen for which there is a memory T cell population in peripheral blood. EBV is, however, of particular interest as a candidate pathogen in MS. EBV infection is almost a prerequisite for MS development, and perturbation of the immune response against EBV with increasing anti-EBNA IgG levels seems to precede the development of MS (Ascherio et al., Clin Exp Immunol. 2010; 160(1):120-124). The mechanisms that direct EBV-reactive T cells to the CSF is, however, not clear. Latently and lytically EBV-infected B cells as well as EBV-encoded RNA (EBER)⁺ B cells have been found in MS brains (Serafini B, et al. J Exp Med. 2007; 204(12):2899-2912; Serafini et al., Brain. 2013; 136(Pt 7):e233), indicating that accumulation of EBV-reactive T cells might be part of an immune response to a chronic EBV infection in the MS brain. Others have, however, not been able to reproduce these results (Willis S N, et al. Brain. 2009; 132(pt 12):3318-3328; Peferoen L A, et al. Brain. 2010; 133(pt 5):e137; Torkildsen O, et al. Brain Pathol. 2010; 20(4):720-729; Sargsyan S A, et al. Neurology. 2010; 74(14):1127-1135). Nevertheless, a recent study has demonstrated EBER⁺ B cells associated with innate immune activation in active MS lesions, and also in two cases of stroke (Tzartos J S, et al. Neurology. 2012; 78(1):15-23). This could indicate that EBV infection, and the subsequent immune response, may contribute to neuroinflammation in MS and possibly also other CNS diseases.

EBV antigens are in vivo presented to T cells by EBV-infected B cells displaying both lytic and latent antigens (Bhaduri-McIntosh S, et al., Blood. 2008; 111(3):1334-1343). In line with others (Cepok S, et al. J Clin Invest. 2005; 115(5):1352-1360; Pender M P, et al., J Neurol Neurosurg Psychiatry. 2009; 80(5):498-505; Lindsey J W, et al., J Neuroimmunol. 2010; 229(1-2):238-242), it was determined that autologous EBV-LCL would represent a broader and more physiological stimulus for the EBV-specific T cell repertoire than selected EBV proteins or peptides.

The presence of EBV-reactive T cells in CSF of MS patients has been described (Holmoy T, et al; J Neurovirol. 2004; 10(1):52-56; Lossius A, et al., J Neuroimmunol. 2011; 240-241: 87-96; Jaquiery E, et al. Eur J Immunol. 2010; 40(3):878-887). By measurements of cytotoxic T cell responses in a large cohort of patients, Jaquiéry et al showed that CD8⁺ T cells display a far higher EBV-specific cytolytic activity in the CSF than in blood of MS patients, whereas the opposite was found for OND patients (Jaquiery et al., supra).

A public TCR or TCR domain displays sequence homology shared among multiple individuals and characteristically shows clonal dominance within the immune response (Venturi et al., Nat Rev Immunol. 2008; 8(3):231-238). An increasing number of such public T cell responses have been identified recent years (Venturi et al., supra), including those against myelin epitopes in experimental autoimmune encephalomyelitis, an animal model of MS (Fazilleau N, et al. Eur J Immunol. 2006; 36(3):533-543; Menezes J S, et al. J Clin Invest. 2007; 117(8):2176-2185). It was found that a previously identified HLA-A2-restricted EBV-associated public TCRβ CDR3 sequence (Lim A, et al. J Immunol. 2000; 165(4):2001-2011) was intrathecally enriched in 2 of the 6 HLA-A2 positive MS patients studied here. In fact, this sequence has previously been identified as monoclonal expansions in MS lesions, although the authors did not comment on the specificity (Junker A, et al. Brain. 2007; 130(pt 11):2789-2799), and is associated with a public CD8⁺ T cell response directed against the epitope GLCTLVAML (SEQ ID NO: 253) from the lytic EBV protein BMLF1 (Lim A, et al. J Immunol. 2000; 165(4):2001-2011). This and the other EBV-specific public TCRs were all enriched in the CSF of three MS patients, whereas such sequences were more prevalent in blood of two controls. Although from a restricted number of subjects, these results support those showing a selective increase in the total repertoire of EBV-reactive CD8⁺ T cells in the CSF of MS patients.

As an exemplary application of the technology, experiments were conducted to characterize the TCR gene frequencies in the disease MS. MS is mediated by an immunological attack on the brain involving T cells. High-throughput sequencing was used to characterize the T cell receptor (TCR)β CDR3 genes on paired samples of cerebrospinal fluid (CSF) and blood from MS patients and controls with other neuroinflammatory diseases.

The results indicated that TCR repertoires were equally diverse in both compartments without preferential Vβ or Jβ-gene usage in patients and controls. Clones were differentially expanded in CSF and blood from patients, and remained expanded in CSF for at least 14 months. Intrathecally accumulated EBV-reactive CD4+ TCRs were found to some extent in both patients and controls.

However, EBV-reactive CD8+ TCRs, including a public TCR previously derived from MS lesions, were enriched in CSF of MS patients only. This study indicates that a clonally diverse, yet stable and compartmentalized, intrathecal T cell response is associated with (and provides a diagnostic marker for) MS. As a result, embodiments of the technology identified and quantified EBV-reactive TCRs in CSF using sorted EBV-responding T cells from autologous blood as a reference.

Accordingly, embodiments of the present disclosure provide methods for identifying disease related antigen from T cell receptor genes. While the present disclosure is exemplified with MS, the present disclosure is not limited to a particular disease. The methods and systems described herein find use in the identification of disease related antigens from a variety of inflammatory, immune, autoimmune, cancer, genetic, fibrotic, infectious, and other diseases.

In some embodiments, the methods comprise the steps of sequencing a set of T cell receptor genes from disease and control sample, and comparing the T cell receptor gene frequencies of the set of T cell receptor genes from the disease-related sample with the T cell receptor gene frequencies of the set of T cell receptor genes from the control sample to identify T cell receptor genes enriched in the disease-related sample. In some embodiments, a set of one or more (e.g., 10 or more, 50 or more, 100 or more, or 500 or more) genes are identified.

In some embodiments, antigens are identified by contacting a control sample from a subject diagnosed with a disease (e.g., inflammatory or autoimmune disease) with a test antigen and a control antigen; isolating T cells from the control sample that respond to said test antigen to provide control sample test-antigen responsive T cells and isolating T cells from the control sample that respond to said control antigen to provide control sample control-antigen responsive T cells; sequencing T-cell receptor genes from said control sample test-antigen responsive T cells to provide control sample test-antigen responsive gene sequences and sequencing T-cell receptor genes from said control sample control-antigen responsive T cells to provide control sample control-antigen responsive gene sequences; isolating T cells from the disease-related sample to provide disease-related T cells; sequencing T-cell receptor genes from said disease-related T cells to provide disease related gene sequences; and comparing the frequency of said control sample test-antigen responsive gene sequences and control sample control-antigen responsive gene sequences with the frequency of said disease-related gene sequences, wherein the gene sequences enriched in the disease-related sample identify a disease-related antigen associated with the disease.

The antigens described herein (e.g. in Tables 3-5) and identified using the methods described herein find use in a variety of applications. Examples include, but are not limited to, use in immunotherapy.

In some embodiments, the present disclosure provides therapeutic agents that target the antigens described herein (e.g., to enhance or minimize an immune response to the antigen). In some embodiments, agents are e.g., small molecules, antibodies, nucleic acids, aptamers, etc. In some embodiments, the present disclosure provides agents (e.g., antibodies or aptamers) that interact with antigens described herein and minimize the immunological (e.g., autoimmune) response to the antigen.

In some embodiments, the antigens, including fragments, derivatives and analogs thereof, may be used as immunogens to produce antibodies having use in the diagnostic, screening, research, and therapeutic methods described herein. The antibodies may be polyclonal or monoclonal, chimeric, humanized, single chain, Fv or Fab fragments. Various procedures can be used for the production and labeling of such antibodies and fragments. See+, e.g., Burns, ed., Immunochemical Protocols, 3rd ed., Humana Press (2005); Harlow and Lane, Antibodies: A Laboratory Manual, Cold Spring Harbor Laboratory (1988); Kozbor et al, Immunology Today 4: 72 (1983); Köhler and Milstein, Nature 256: 495 (1975).

Aptamers are a class of molecule that represents an alternative to antibodies in term of molecular recognition. Aptamers are oligonucleotide or oligopeptide sequences with the capacity to recognize virtually any class of target molecules with high affinity and specificity. Such ligands may be isolated through Systematic Evolution of Ligands by EXponential enrichment (SELEX) of a random sequence library, as described in Tuerk C. and Gold L., 1990. The random sequence library is obtainable by combinatorial chemical synthesis of DNA. In this library, each member is a linear oligomer, eventually chemically modified, of a unique sequence. Possible modifications, uses and advantages of this class of molecules have been reviewed in Jayasena S. D., 1999. Peptide aptamers consists of a conformationally constrained antibody variable region displayed by a platform protein, such as E. coli Thioredoxin A that are selected from combinatorial libraries by two hybrid methods (Colas et al., 1996).

In some embodiments, the present disclosure provides pharmaceutical compositions that target antigens identified using the methods described herein, In some embodiments, compositions comprise sterile aqueous preparations. Acceptable vehicles and solvents include, but are not limited to, water, Ringer's solution, phosphate buffered saline, and isotonic sodium chloride solution. In addition, sterile, fixed oils are conventionally employed as a solvent or suspending medium. For this purpose any bland fixed mineral or non-mineral oil may be employed including synthetic mono-ordi-glycerides. In addition, fatty acids such as oleic acid find use in the preparation of injectables. Carrier formulations suitable for mucosal, subcutaneous, intramuscular, intraperitoneal, intravenous, or administration via other routes may be found in Remington's Pharmaceutical Sciences, Mack Publishing Company, Easton, Pa.

Other delivery systems include time-release, delayed release, or sustained release delivery systems. Such systems can avoid repeated administrations of the compositions, increasing convenience to the subject and a physician. Many types of release delivery systems are available and known to those of ordinary skill in the art. They include polymer based systems such as poly(lactide-glycolide), copolyoxalates, polycaprolactones, polyesteramides, polyorthoesters, polyhydroxybutyric acid, and polyanhydrides. Microcapsules of the foregoing polymers containing drugs are described in, for example, U.S. Pat. No. 5,075,109, hereby incorporated by reference. Delivery systems also include non-polymer systems that are: lipids including sterols such as cholesterol, cholesterol esters and fatty acids or neutral fats such as mono-di- and tri-glycerides; hydrogel release systems; sylastic systems; peptide based systems; wax coatings; compressed tablets using conventional binders and excipients; partially fused implants; and the like. Specific examples include, but are not limited to: (a) erosional systems in which an agent of the invention is contained in a form within a matrix such as those described in U.S. Pat. Nos. 4,452,775, 4,675,189, and 5,736,152, each of which is hereby incorporated by reference and (b) diffusional systems in which an active component permeates at a controlled rate from a polymer such as described in U.S. Pat. Nos. 3,854,480, 5,133,974 and 5,407,686, each of which is hereby incorporated by reference. In addition, pump-based hardware delivery systems can be used, some of which are adapted for implantation.

In some embodiments, a composition of the present disclosure is formulated in a concentrated dose that can be diluted prior to administration to a subject. For example, dilutions of a concentrated composition may be administered to a subject such that the subject receives any one or more of the specific dosages provided herein. In some embodiments, dilution of a concentrated composition may be made such that a subject is administered (e.g., in a single dose) a composition comprising 0.5-50% of a material present in the concentrated composition. Concentrated compositions are contemplated to be useful in a setting in which large numbers of subjects may be administered a composition of the present invention (e.g., an immunization clinic, hospital, school, etc.). In some embodiments, a composition (e.g., a concentrated composition) is stable at room temperature for more than 1 week, in some embodiments for more than 2 weeks, in some embodiments for more than 3 weeks, in some embodiments for more than 4 weeks, in some embodiments for more than 5 weeks, and in some embodiments for more than 6 weeks.

The pharmaceutical compositions of the present disclosure may be administered in a number of ways depending upon whether local or systemic treatment is desired and upon the area to be treated. Administration may be topical (including ophthalmic and to mucous membranes including vaginal and rectal delivery), pulmonary (e.g., by inhalation or insufflation of powders or aerosols, including by nebulizer; intratracheal, intranasal, epidermal and transdermal), oral or parenteral. Parenteral administration includes intravenous, intraarterial, subcutaneous, intraperitoneal or intramuscular injection or infusion; or intracranial, e.g., intrathecal or intraventricular, administration. A continuous delivery of the drug for a longer period of time is achieved by formulating the drug in, for example, a polymer or other device that releases drug constantly at the administration site or target.

Pharmaceutical compositions and formulations for topical administration may include transdermal patches, ointments, lotions, creams, gels, drops, suppositories, sprays, liquids and powders. Conventional pharmaceutical carriers, aqueous, powder or oily bases, thickeners and the like may be necessary or desirable.

Compositions and formulations for oral administration include powders or granules, suspensions or solutions in water or non aqueous media, capsules, sachets or tablets. Thickeners, flavoring agents, diluents, emulsifiers, dispersing aids or binders may be desirable.

Compositions and formulations for parenteral, including intravenous, intramuscular and subcutaneous, or intrathecal or intraventricular administration may include sterile aqueous solutions that may also contain buffers, diluents and other suitable additives such as, but not limited to, penetration enhancers, carrier compounds and other pharmaceutically acceptable carriers or excipients.

Pharmaceutical compositions of the present disclosure include, but are not limited to, solutions, emulsions, micelle and liposome containing formulations. These compositions may be generated from a variety of components that include, but are not limited to, preformed liquids, self emulsifying solids and self emulsifying semisolids. In some embodiments, compounds are formulated as extended release compounds (e.g., in a polymer base for injection).

The pharmaceutical formulations of the present disclosure, which may conveniently be presented in unit dosage form, may be prepared according to conventional techniques well known in the pharmaceutical industry. Such techniques include the step of bringing into association the active ingredients with the pharmaceutical carrier(s) or excipient(s). In general the formulations are prepared by uniformly and intimately bringing into association the active ingredients with liquid carriers or finely divided solid carriers or both, and then, if necessary, shaping the product.

The compositions of the present disclosure may be formulated into any of many possible dosage forms such as, but not limited to, tablets, capsules, sterile parenteral solutions, liquid syrups, soft gels, suppositories, and enemas. The compositions of the present disclosure may also be formulated as suspensions in aqueous, non aqueous or mixed media. Aqueous suspensions may further contain substances that increase the viscosity of the suspension including, for example, sodium carboxymethylcellulose, sorbitol and/or dextran. The suspension may also contain stabilizers.

In one embodiment of the present disclosure the pharmaceutical compositions may be formulated and used as foams. Pharmaceutical foams include formulations such as, but not limited to, emulsions, microemulsions, creams, jellies and liposomes. While basically similar in nature these formulations vary in the components and the consistency of the final product.

The compositions of the present disclosure may additionally contain other adjunct components conventionally found in pharmaceutical compositions. Thus, for example, the compositions may contain additional, compatible, pharmaceutically active materials such as, for example, antipruritics, astringents, local anesthetics or anti inflammatory agents, or may contain additional materials useful in physically formulating various dosage forms of the compositions of the present disclosure, such as dyes, flavoring agents, preservatives, antioxidants, opacifiers, thickening agents and stabilizers. However, such materials, when added, should not unduly interfere with the biological activities of the components of the compositions of the present disclosure. The formulations can be sterilized and, if desired, mixed with auxiliary agents, e.g., lubricants, preservatives, stabilizers, wetting agents, emulsifiers, salts for influencing osmotic pressure, buffers, colorings, flavorings and/or aromatic substances and the like which do not deleteriously interact with the active agents of the formulation.

In some embodiments, following an initial administration of a composition, a subject may receive one or more additional administrations (e.g., around 2 weeks, around 3 weeks, around 4 weeks, around 5 weeks, around 6 weeks, around 7 weeks, around 8 weeks, around 10 weeks, around 3 months, around 4 months, around 6 months, around 9 months, around 1 year, around 2 years, around 3 years, around 5 years, around 10 years) subsequent to a first, second, third, fourth, fifth, sixth, seventh, eighth, ninth, tenth, and/or more than tenth administration.

Dosage units may be proportionately increased or decreased based on several factors including, but not limited to, the weight, age, and health status of the subject. In addition, dosage units may be increased or decreased for subsequent administrations (e.g., boost administrations). It is contemplated that the compositions and methods of the present invention will find use in various settings, including research settings. For example, compositions and methods of the present invention also find use in studies of the immune system (e.g., characterization of adaptive immune responses (e.g., protective immune responses (e.g., mucosal or systemic immunity))). Uses of the compositions and methods provided by the present invention encompass human and non-human subjects and samples from those subjects, and also encompass research applications using these subjects. Thus, it is not intended that the present invention be limited to any particular subject and/or application setting.

The present disclosure further provides kits comprising the compositions comprised herein. In some embodiments, the kit includes all of the components necessary, sufficient or useful for administering an agent that targets the antigen. For example, in some embodiments, the kits comprise devices for administering the composition (e.g., needles or other injection devices), temperature control components (e.g., refrigeration or other cooling components), sanitation components (e.g., alcohol swabs for sanitizing the site of injection) and instructions for administering the composition.

In this detailed description of the various embodiments, for purposes of explanation, numerous specific details are set forth to provide a thorough understanding of the embodiments disclosed. In addition, the section headings used are for organizational purposes only and are not to be construed as limiting the described subject matter in any way. One skilled in the art will appreciate, however, that these various embodiments may be practiced with or without these specific details. In other instances, structures and devices are shown in block diagram form. Furthermore, one skilled in the art can readily appreciate that the specific sequences in which methods are presented and performed are illustrative and it is contemplated that the sequences can be varied and still remain within the spirit and scope of the various embodiments disclosed herein.

All literature and similar materials cited in this application, including but not limited to, patents, patent applications, articles, books, treatises, and internet web pages are expressly incorporated by reference in their entirety for any purpose. Unless defined otherwise, all technical and scientific terms used herein have the same meaning as is commonly understood by one of ordinary skill in the art to which the various embodiments described herein belongs. When definitions of terms in incorporated references appear to differ from the definitions provided in the present teachings, the definition provided in the present teachings shall control.

### EXAMPLES

### Methods

### Subjects.

Patients with MS and OND were recruited at the Departments of Neurology at Oslo University Hospital and Akershus University Hospital. All subjects were included as they underwent lumbar puncture during diagnostic workup, except MS-9, who participated solely for research. A diagnosis of MS satisfied the revised McDonald criteria (61). None of the MS patients used disease-modifying drugs at inclusion, and only one (MS-9) had used such drugs previously. All patients and controls were EBV seropositive, determined by the presence of serum IgG antibodies against EBV nuclear antigen 1 and/or viral capsid antigen.

### Sample Acquisition and Preparation.

Between 20 and 25 ml of CSF were collected by lumbar puncture. The first 2 ml were discarded to eliminate possible blood contamination. After centrifugation at 500×g in 10 minutes, the supernatant was carefully removed, and the pellet was dissolved in the remaining 100 μl of CSF. A volume of 10 μl was examined microscopically for cell count and blood contamination in a Bürker chamber (VWR). The CSF sample of OND-8 contained a minute contamination of 3 red blood cells (RBC) per 15 mononuclear cells. None of the other 20 CSF samples contained any visible RBC. The cell suspension was transferred to a 2-ml Eppendorf tube and spun down at 16,100×g for 7 minutes. The supernatant was carefully removed and the pellet was snap frozen in liquid nitrogen. PBMC and serum were harvested the day of the lumbar puncture. PBMC were isolated from whole blood using gradient separation with Lymphoprep (Axis-Shield). 4.0×10⁶ PBMC were transferred to a 2-ml Eppendorf tube, spun down, and snap frozen as described above. The remaining PBMC were frozen in 10% DMSO and stored in liquid nitrogen. For the majority of the snap frozen samples, total genomic DNA was extracted using the QIAamp DNA Micro Kit or the QIAamp DNA Blood Mini Kit (Qiagen) according to manufacturer instructions. For samples from MS-1, MS-2, MS-3, MS-4, MS-5, OND-1, and OND-2, the PCR was done directly on cell lysates. The number of mononuclear CSF cells obtained varied from 20,000 to 150,000. For the PBMC samples, the DNA concentration was normalized to correspond to 200,000 cell genomes.

### Antigens.

5.0×10⁶ PBMC from each subject were used to generate EBV-LCL by culturing cells in Gibco RPMI 1640 supplemented with 5% fetal calf serum and supernatant from a B95-8 EBV-infected marmoset cell line. Ciclosporin (Novartis) was added at a concentration of 0.5 μg/ml the next day and used in the medium the two first weeks of culture. All cultures were examined microscopically at regular intervals and acquired a typical blast-like appearance within 1 to 2 weeks. The cells were used in stimulation assays after 2 to 4 months of culture. Three days prior to assay, the medium was changed to Gibco RPMI 1640 supplemented with 10% heat inactivated autologous serum. Influenza A virus (X-31, A/Aichi/68; H3N2) was purchased from Charles River Laboratories, Wilmington, Mass., USA. A live, attenuated VZV vaccine was also used (Varilrix; GlaxoSmithKline).

### Isolation of Virus-Reactive T Cells.

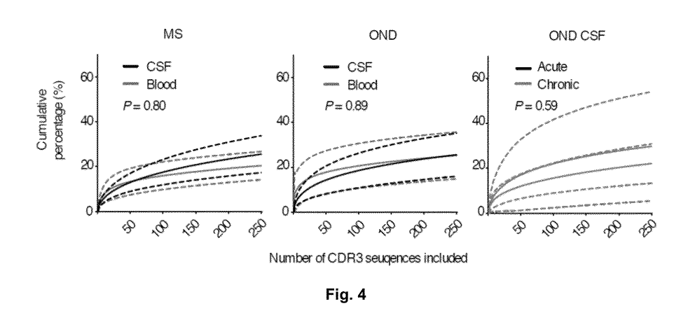

6.0×10⁷ PBMC were thawed and stained with 0.5 μM CFDA-SE (Invitrogen) for 8 minutes at room temperature. The mononuclear cells were washed, resuspended in Gibco RPMI 1640 supplemented with 10% heat inactivated autologous serum, and 2.5×10⁷ cells were transferred to two separate wells in a 6-well plate (Corning). To these wells, either irradiated (80 Gy) autologous EBV-LCL at a stimulator-to-responder ratio of 1:5 or Influenza A virus at 50,000 hemagglutinin units (HAU)/ml was added. Unstained and stained unstimulated PBMC at 1.0×10⁶ cells per well in a 96-well flat bottom plate (Corning) served as negative and compensation controls. After 8 days of incubation at 37° C. in 5% CO₂, another 2.5×10⁷ PBMC were thawed, stained with CellTrace Violet (Invitrogen, Molecular probes) at 0.5 μM in 20 minutes, and incubated overnight with influenza A at 50,000 HAU/ml. The next day, 1.0×10⁶ cells were transferred from the primary cell cultures to wells in the 96-well flat bottom plates as additional controls. The primary cultures were then restimulated with either novel influenza infected PBMC or irradiated autologous EBV-LCL as described above. The negative control was restimulated with 1.0×10⁶ uninfected PBMC. Twenty-four hours after restimulation, the cells were harvested and labeled with a mixture of fluorochrome-conjugated antibodies purchased from BD Biosciences: CD137-PE (4B4-1), CD14-PerCP (MφP9), CD20-PerCP Cy5.5 (2H7), CD8-APC(RPA-T8), and CD4-PE-Cy7 (SK3). Propidium iodide (BD Biosciences) was added immediately before the acquisition to exclude dead cells. CFSE1owCD137+ CD4+ and CFSE1owCD137+ CD8+ subsets were sorted on a FACSAria flow cytometer (BD Biosciences) equipped with 405-nm, 488-nm, and 633-nm lasers as shown in FIG. 4A. The T cells of OND-10 did not respond to stimulation with influenza A virus. CD4+ and CD8+ T cells from all other subjects reacted upon stimulation with both antigens—5,000 to 26,000 CD4+, 10,000 to 60,000 CD8+ EBV-reactive T cells, 2,500 to 20,000 CD4+, and 4,000 to 50,000 CD8+ Influenza A reactive T cells were obtained. Due to low frequencies of influenza-responding CD4+ T cells of MS-8 and MS-9, the entire CFSE1owCD4+ populations were sorted in these cases (irrespective of CD137 status). Genomic DNA was extracted from sorted cell samples as described above.

### High-Throughput Sequencing of the TCRβ Chains.

TCRβ sequencing was performed by Adaptive Biotechnologies using the TCRB survey level assay for both CSF and virus-reactive T cell subsets and the TCRB deep level assay for PBMC samples (Robins H S, et al. Blood. 2009; 114(19):4099-4107). Two blinded samples having T cell clones of known TCRβ CDR3 sequences sorted in a polyclonal T cell population were used as quality controls. Two or more aliquots of three PBMC samples were sequenced at different time points to verify reproducibility and to assess the limitations in sequence coverage due to finite sampling. To assess and remove PCR bias from the multiplex PCR assay, a synthetic immune system with all possible V-J combinations was precisely quantified. These synthetic templates were used to calibrate the assay and normalize the output.

### TCRβ Sequence Data Processing.

All sequence data were processed and uploaded into the ImmunoSEQ Analyzer at Adaptive Biotechnologies. The processing includes PCR and sequencing error correction (Robins H S, et al. supra). The TCRβ CDR3 region was defined according to the International ImMunoGeneTics (IMGT) collaboration, and the Vβ-, Dβ- and Jβ-genes of each CDR3 sequence were determined using the IMGT/JunctionAnalysis algorithm (Yousfi Monod M, et al., Bioinformatics. 2004; 20(suppl 1):S379-S385). Complete identity between the deduced amino acid sequences of TCRβ CDR3 regions was required for a sequence match between samples. For all individuals tested, a small proportion of the CDR3 sequences present in the sample stimulated with EBV-LCL were also present in the sample stimulated with influenza A virus. Such sequences were regarded as unspecific and removed from further analyses. There was also a small overlap of CDR3 sequences between samples of sorted CD4+ and CD8+ T cells reactive upon stimulation with the same antigen. These sequences were assigned to either sample according to their relative frequencies.

### Statistics.

The number of CDR3 sequences is presented as the mean with 95% CI, or as a the median and range if the distribution was skewed. To assess the degree of similarity between clonal populations in CSF and blood, the Morisita-Horn index (Venturi et al., supra) and Bland-Altman plots (Bland J M, Altman D G. Lancet. 1986; 1(8476):307-310) were used. Friedman's 2-way ANOVA by ranks test was used when comparing the distribution of sequences in CSF with that in blood and to compare blood and CSF with regard to Vβ or Jβ-chain usage. The Wilcoxon signed rank test was used to compare the frequencies of EBV-reactive T cells in CSF with blood. The Mann-Whitney U test was used to compare the frequency distributions between independent groups, sequences in the CSF of acute vs. chronic CNS inflammation, Vβ or Jβ-chain usage in CSF from patients vs. controls, and the Morisita-Horn indexes of CSF and blood vs. serial blood draws. The average areas under the individual curves of cumulative percentages of the 250 most frequent clones were calculated by repeatedly applying the area formula for a trapezium. To compare the impact of these clones in blood and CSF, and in CSF of controls with acute vs. chronic inflammation, the average areas were compared employing paired or independent samples t-test, respectively. Pearson's correlation coefficient (r) was calculated to quantify the association between CDR3 frequencies at two time points for the same patient. Level of significance was set at 5%, and all tests were two-sided. The analyses were performed in IBM SPSS Statistics version 20 and GraphPad Prism 6 for Mac OS X. Flow cytometry data used in FIG. 4A are analyzed in FlowJo version 10.0.7 for Mac OS X.

### Study Approval.

The study was approved by the Committee for Research Ethics at the South-Eastern Norwegian Health Authority (2009/23 S-04143a), and all participants gave written informed consent before inclusion.

### Example 1

### Intrathecal T Cells in MS and OND Contain Expanded Clones that Constitute a Low Proportion of Highly Diverse T Cell Repertoires

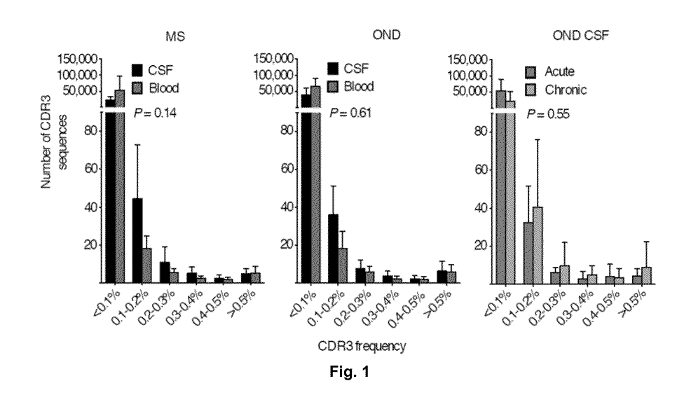

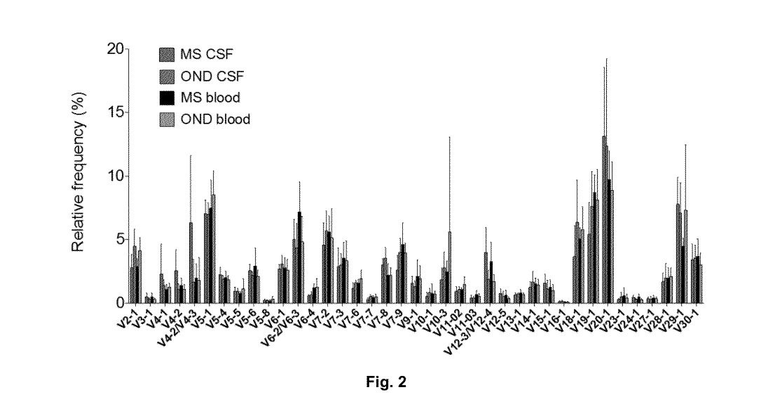

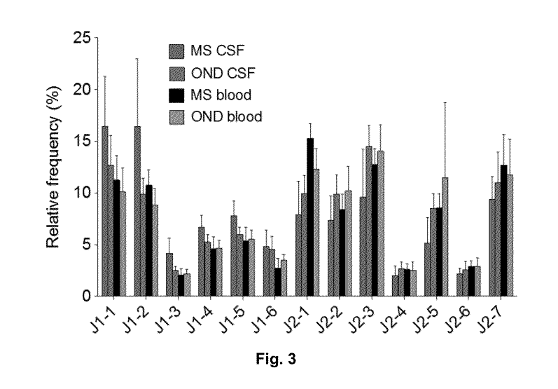

During the development of embodiments of the technology provided herein, experiments were conducted to characterize the intrathecal T cell response in MS. In particular, data were collected to compare the TCRβ-chain repertoires of mononuclear cells from paired CSF and blood samples harvested from 10 patients with MS and 11 controls with OND. Subject and disease characteristics and HLA-DRB1, -A and -B types are listed in Table 1. From the MS patients, an average of 22,200 (95% confidence interval (CI)±9, 450) unique productive sequences were obtained from the CSF and 52,100 (95% CI±35,100) were obtained from blood (Table 2). The corresponding numbers from the CSF and blood of controls were 38,400 (95% CI±18,300) and 63,900 (95% CI±20,800), respectively. In accordance with the large number of sequences obtained from CSF and blood in both patients and controls, the frequency distributions in both compartments were strongly right-skewed, with a large majority of CDR3 sequences present in copy numbers below 0.1% of the total number of sequences (FIG. 1A). The frequency distribution in CSF was not significantly different from that in blood, neither for MS patients nor for controls with OND. Similar distributions were also observed in the CSF of the controls with acute as compared with chronic neuroinflammation (FIG. 1). The analysis did not indicate the presence of a biased Vβ or Jβ-gene usage in blood or CSF, but showed similar distributions between compartments and between patients and controls (FIGS. 2 and 3).

The top clones in each compartment, here defined as unique CDR3 sequences present at copy numbers above 0.1% of the total repertoire, represent expanded T cell clones. The MS patients had a median of 43.5 (range 30 to 214) clones present at frequencies above 0.1% in the CSF and 36 (range 14 to 49) in blood. The corresponding numbers for subjects with OND were 50 (range 11 to 148) and 31 (range 8 to 90). To explore how much expanded clones contribute to the total TCR repertoire in CSF and blood of patients and controls, calculations were performed to find the average area under the individual curves representing cumulative percentages of the 250 most frequent CDR3 sequences in CSF and blood (FIG. 4). There was no statistically significant difference between CSF and blood from patients or controls, or between the average areas representing CSF clones from controls with acute and chronic neuroinflammation. Collectively, the results indicated that expanded T cell clones constitute a low fraction of highly diverse TCR repertoires in the CSF of patients with MS as well as OND, and that the clonal distributions and repertoire diversities in the CSF and blood are similar.

TABLE 1
Subject and disease characteristics

| | | | | Disease | | CSF | HLA- | | |
|---|---|---|---|---|---|---|---|---|---|
| | | Age | | duration | | cell | DRB | HLA- | HLA- |
| ID | Sex | (yrs) | Diagnosis | (months) | OBC | count | 1* | A* | B* |
| MS-1 | F | 31 | RR-MS | 2 | + | 25 | 07, | 25, | 18, |
| | | | | | | | 08 | 31 | 44 |
| MS-2 | F | 38 | RR-MS | 1 | + | 7 | 15 | 02, | 07, |
| | | | | | | | | 03 | 37 |
| MS-3 | M | 39 | RR-MS^{A} | 8 | + | 15 | 13, | 02, | 07, |
| | | | | | | | 15 | 24 | 40 |
| MS-4 | F | 20 | RR-MS^{A} | 11 | + | 2 | 04, | 03, | 15, |
| | | | | | | | 13 | 68 | 39 |
| MS-5 | F | 45 | CIS | 9 | + | 7 | 15 | 02, | 07, |
| | | | | | | | | 03 | 18 |
| MS-6 | F | 29 | RR-MS | 16 | + | 12 | 15 | 01, | 07, |
| | | | | | | | | 24 | 18 |
| MS-7 | M | 37 | RR-MS^{A} | 6 | + | 15 | 11 | 02, | 18, |
| | | | | | | | | 25 | 44 |
| MS-8 | M | 29 | RR-MS | 4 | + | 11 | 03, | 01, | 08, |
| | | | | | | | 15 | 03 | 35 |
| MS-9 | F | 33 | RR-MS | 12 | + | 5 | 07, | 02 | 14, |
| | | | | | | | 15 | | 44 |
| MS-10 | F | 32 | RR-MS | 60 | + | 10 | 04, | 02, | 18, |
| | | | | | | | 15 | 25 | 27 |
| OND-1 | M | 45 | Aseptic meningitis | 1 | − | 17 | 03, | 01, | 08, |
| | | | | | | | 15 | 68 | 35 |
| OND-2 | M | 39 | VZV | 2 | − | 5 | 07, | 01, | 44 |
| | | | meningoencephalitis | | | | 11 | 32 | |
| OND-3 | M | 28 | Aseptic | 0 | − | 40 | 03, | 01, | 08, |
| | | | meningoencephalitis | | | | 07 | 02 | 50 |
| OND-4 | F | 30 | Transverse myelitis | 1 | − | 24 | 04, | 02 | 07, |
| | | | | | | | 14 | 02 | 15 |
| OND-5 | F | 58 | ADEM | 0 | − | 20 | 03, | 01, | 08, |
| | | | | | | | 04 | 02 | 44 |
| OND-6 | M | 39 | Neurosarcoidosis | 13 | − | 75 | 03 | 01 | 08 |
| OND-7 | M | 42 | Polyradiculitis | 7 | + | 10 | 01, | 02 | 15, |
| | | | | | | | 04 | | 27 |
| OND-8 | F | 45 | Aseptic meningitis | 0 | − | 14 | 07, | 03, | 07, |
| | | | | | | | 15 | 68 | 44 |
| OND-9 | M | 48 | Neurosarcoidosis | 16 | − | 20 | 03 | 01 | 08 |
| OND- | M | 40 | Neurosarcoidosis | 3 | − | 8 | 04, | 03, | 07, |
| 10 | | | | | | | 15 | 32 | 44 |
| OND- | F | 60 | Inflammatory cranial | 6 | − | 6 | 03, | 01, | 07, |
| 11 | | | neuropathy | | | | 15 | 02 | 08 |

The disease duration is calculated from the timepoint of the first disease symptom. Oligoclonal bands (OCB) positive (+) indicates the presence of >2 IgG bands on isoelectric focusing of cerebospinal fluid (CSF) that was not found in serum. CSF cell count denotes the number of mononuclear cells per microliter CSF. A Clinically isolated syndrom (CIS) at inclusion, but later developed definite MS. F, female; M, male; RR-MS, relapsing-remitting MS; ADEM, Acute Demyeliniating Encephalomyelitis; VZV, varicella zoster virus.

TABLE 2
Summary of productive TCRβ CDR3 sequences obtained from the
cerebrospinal fluid (CSF) and blood from each study subject.

| | CSF | | Blood | |
|---|---|---|---|---|
| | Total | Unique | Total | Unique |
| MS-1 | 4,968,110 | 55,428 | 2,973,206 | 30,550 |
| MS-2 | 4,272,636 | 3,200 | 211,883 | 7,365 |
| MS-3 | 6,520,027 | 20,052 | 233,841 | 9,138 |
| MS-4 | 7,618,800 | 19,292 | 1,009,649 | 14,960 |
| MS-5 | 7,877,374 | 40,298 | 2,155,363 | 10,094 |
| MS-6 | 1,780,093 | 13,171 | 2,454,256 | 31,797 |
| MS-7 | 1,161,612 | 16,629 | 5,176,825 | 85,525 |
| MS-8 | 1,553,749 | 11,149 | 5,950,035 | 85,305 |
| MS-9 | 1,333,129 | 16,266 | 6,561,692 | 57,468 |
| MS-10 | 3,926,670 | 26,354 | 4,668,226 | 188,877 |
| OND-1 | 4,330,148 | 59,627 | 7,811,095 | 16,426 |
| OND-2 | 5,694,937 | 30,663 | 6,459,855 | 16,072 |
| OND-3 | 2,710,304 | 72,587 | 1,554,852 | 53,100 |
| OND-4 | 2,611,033 | 101,224 | 1,362,441 | 61,132 |
| OND-5 | 3,249,232 | 29,649 | 838,946 | 29,052 |
| OND-6 | 1,810,248 | 62,761 | 6,263,135 | 128,625 |
| OND-7 | 1,203,853 | 10,260 | 2,972,613 | 67,238 |
| OND-8 | 1,738,143 | 15,113 | 4,684,047 | 79,736 |
| OND-9 | 1,514,126 | 17,171 | 7,643,686 | 67,550 |
| OND-10 | 1,753,206 | 15,835 | 3,364,686 | 77,710 |
| OND-11 | 1,929,633 | 7,326 | 4,572,127 | 106,265 |

### Example 2

### T Cell Clones are Differentially Expanded in CSF and Blood, and Persist in CSF for More than a Year

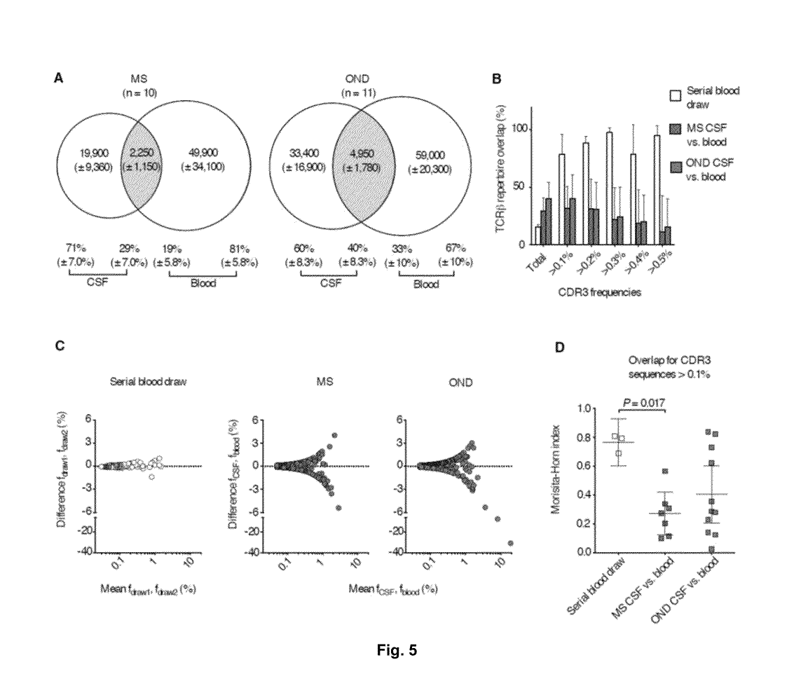

During the development of embodiments of the technology described, experiments were conducted to assess the overlap between the TCRβ repertoires in CSF and blood (FIG. 5A). The shared CDR3 sequences constituted on average 29% (95% CI±7.0%) of the total number of unique sequences in CSF and 19% (95% CI±5.8%) in blood of MS patients, and these numbers were of similar magnitudes among the controls with an average of 40% (95% CI±8.3%) and 33% (95% CI±10%; FIG. 5A). The data also showed overlap between CSF and blood in subsets of CDR3 sequences with increasingly higher frequencies (FIG. 5B).

Due to the large number of different TCRs, only relatively frequent CDR3 sequences are expected to be shared between two serial blood samples from the same individual (FIG. 5B). Thus, if identical T cell clones were equally expanded in CSF and blood, the data are expected to show a large proportion of shared CDR3 sequences among those present at highest frequencies. This was not observed for patients with MS or for controls (FIG. 5B). The degree to which CDR3 sequences of frequencies above 0.1% were shared between blood and CSF is illustrated in Bland-Altman plots (Bland et al., supra) (FIG. 5C). In both patients and controls, T cell clones that were selectively accumulated in the CSF were revealed, but the data also indicated that there were clones that predominated in blood. For CDR3 sequences above 0.1%, the overlap between CSF and blood was quantified using the Morisita-Horn index (Venturi et al, supra) and this was compared with the overlap in serial blood draws from the same individual (FIG. 5D). The overlap between CSF and blood was significantly smaller than expected for patients with MS, compatible with a differential expansion of certain T cell clones in blood and CSF. This did not reach statistical significance for the controls with OND (P=0.17), who displayed a greater variation in the degree of overlap between the two compartments (FIG. 5D).

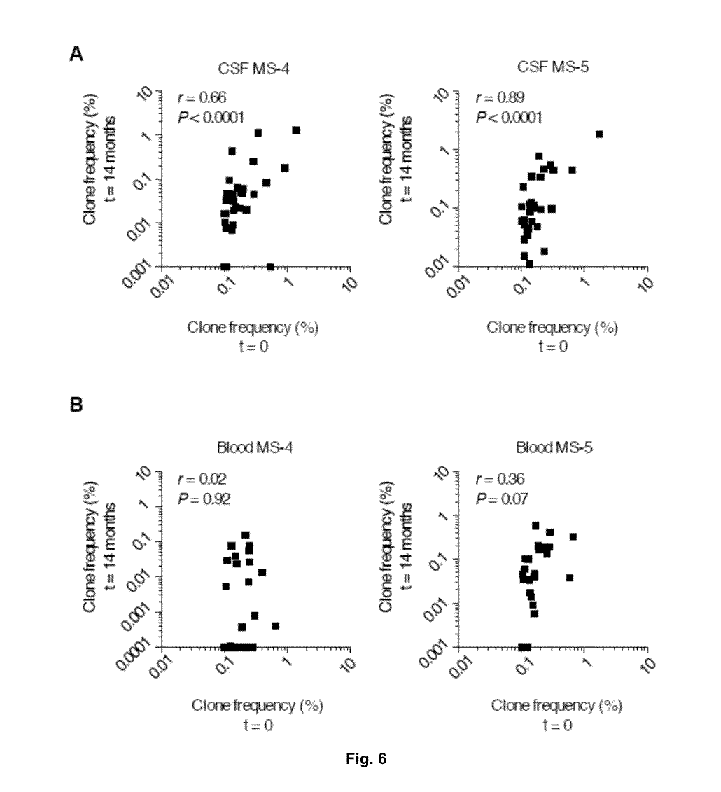

Two patients, MS-4 and MS-5, underwent lumbar punctures separated by 14 months, thus providing samples for experiments to address the persistence of expanded T cell clones in CSF. Among TCRβ CDR3 sequences with frequencies above 0.1% at the first lumbar puncture, 91% (29 of 32; MS-4) and 100% (30 of 30; MS-5) of the sequences were also found in the second CSF sample (FIG. 6A). For both patients, there was a high correlation between the CDR3 frequencies at the two time points. In blood, the corresponding numbers were 47% (16 of 34; MS-4) and 85% (22 of 26; MS-5), and the correlations between the frequencies at the two time points were lower than in CSF (FIG. 6B). Taken together, the results indicate a compartmentalized expansion of T cells, persistent over time, within the CNS of patients with MS.

### Example 3

### CD8+ EBV-Reactive TCRs are Intrathecally Enriched in MS, but not in OND

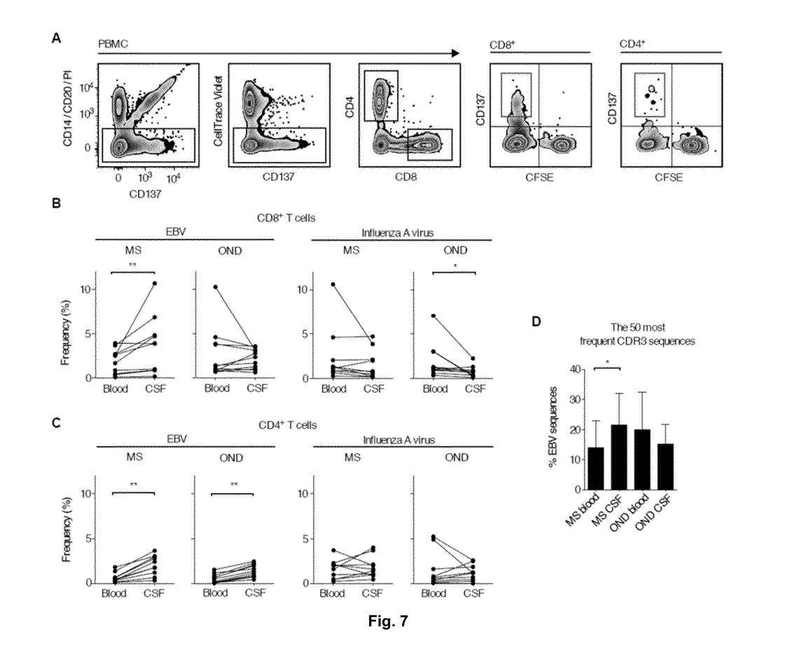

Experiments were conducted during the development of embodiments of the technology provided to determine the contribution of EBV-reactive T cells to the intrathecal T cell repertoire. In particular, experiments were conducted to assess the peripheral blood of EBV-seropositive individuals, which contains large numbers of EBV-reactive T cells (29). Peripheral blood mononuclear cells (PBMC) were stimulated with autologous EBV-lymphoblastoid cell lines (EBV-LCL), and activated CD4+ and CD8+ T cells were sorted to establish reference TCR libraries. Following a similar approach, T cells stimulated with Influenza A virus served as control samples. To maximize antigen specificity, only T cells reacting by proliferation in the first round of antigen stimulation (CFSElow cells), and by the expression of CD137 nine days later following a second stimulation, were sorted (FIG. 4A). Following high-throughput sequencing of the TCRβ CDR3 sequences, the established libraries were used to quantify frequencies of EBV- and influenza A-reactive CD4+ and CD8+ T cells in CSF and blood (FIG. 7B, FIG. 7C, and FIG. 7D).

The estimated frequency of EBV-reactive CD8+ T cells was significantly higher in CSF compared to blood in MS patients (P=0.0059; FIG. 7B), whereas no such enrichment was seen in the OND group. Furthermore, no CSF accumulation was recorded for CD8+ T cells reactive to influenza A virus (FIG. 7B). For EBV-reactive CD4+ T cells, a small but significant increase of estimated frequencies was seen in CSF relative to blood in patients with MS (P=0.002; FIG. 7C), and also in controls with OND (P=0.001). No CSF accumulation of influenza A-reactive CD4+ TCRs was found (FIG. 7C). Finally, EBV-reactive sequences constituted a significantly larger fraction of the 50 most frequent CDR3 sequences in the CSF (21.6%, 95% CI 11.2%-32.0%) than in blood (14.0%, 95% CI 5.1%-22.9%; P=0.023) of MS patients, whereas this was not true for the controls (15.3%, 95% CI 8.7%-21.8% in CSF and 20.0%, 95% CI 5.6%-18.7% in blood; FIG. 7D). The 10 most frequent CDR3 sequences from the CSF of patients and controls, their frequencies in CSF and blood, V- and J-gene usage, and reactivities are listed in Tables 3 and 4.

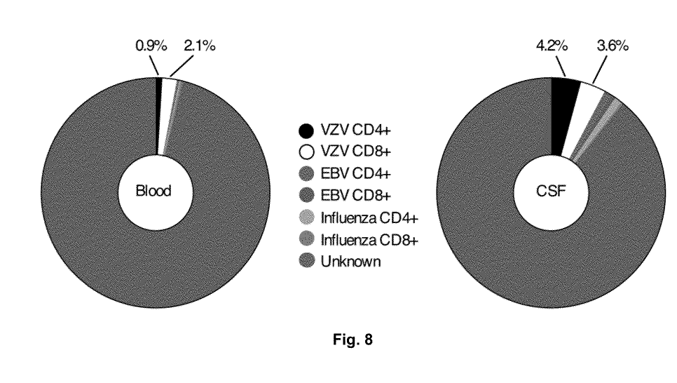

One of the controls (OND-2) had a PCR-verified diagnosis of Varicella zoster virus (VZV) encephalitis. For this individual, it was also estimated the proportion of VZV-reactive CD4+ and CD8+ T cells using the same approach (FIG. 8). In CSF, a total of 7.8% were VZV-reactive (4.2% CD4+ and 3.6% CD8+ T cells), whereas the corresponding number in blood was 3% (0.9% CD4+ and 2.1% CD8+ T cells). In comparison, smaller proportions of the TCRs were reactive to Influenza A virus (1.26% in CSF and 0.42% in blood) or to EBV (1.73% in CSF and 0.39% in blood).

TABLE 3
Frequencies in CSF and blood and V- and J-gene usage of CDR3
sequences from CSF of patients and controls

| | | CSF | Blood | | | Reactivity; | SEQ ID |
|---|---|---|---|---|---|---|---|
| CDR3 | ID | (%) | (%) | V | J | phenotype | NO |
| CASSQDRAGGPYGYTF | MS-1 | 1.76 | 0.07 | 4-3 | 1-2 | | 1 |
| CASSLGRGYTF | MS-1 | 1.53 | 0.04 | 5-6 | 1-2 | EBV; | 2 |
| | | | | | | CD8+ | |
| CASTWGVVAGELFF | MS-1 | 1.25 | 0.31 | 6 | 2-2 | EBV; | 3 |
| | | | | | | CD8+ | |
| CASSPHGGSANVLTF | MS-1 | 1.01 | 0.06 | 4-3 | 2-6 | | 4 |
| CASSPGTDYGYTF | MS-1 | 0.78 | 0.01 | 4-2 | 1-2 | | 5 |
| CASSQHTGIPGNTIYF | MS-1 | 0.68 | 0.01 | 4-1 | 1-3 | EBV; | 6 |
| | | | | | | CD8+ | |
| CSARAERGQHF | MS-1 | 0.52 | 0.02 | 20-1 | 1-5 | EBV; | 7 |
| | | | | | | CD8+ | |
| CASSLAYTGELFF | MS-1 | 0.50 | 0.02 | 4-2 | 2-2 | EBV; | 8 |
| | | | | | | CD8+ | |
| CASSPTQGTFYGYTF | MS-1 | 0.47 | 0.00 | 4 | 1-2 | EBV; | 9 |
| | | | | | | CD8+ | |
| CASTSSPLSTEAFF | MS-1 | 0.44 | 0.01 | 6 | 1-1 | EBV; | 10 |
| | | | | | | CD4+ | |
| CSTPRDNAKNTEAFF | MS-2 | 4.17 | 0.18 | 29-1 | 1-1 | FLU; | 11 |
| | | | | | | CD4+ | |
| CSVGQGGTNEKLFF | MS-2 | 1.63 | 0.04 | 29-1 | 1-4 | EBV; | 12 |
| | | | | | | CD8+ | |
| CSGLKQGGTEAFF | MS-2 | 1.26 | 1.06 | 20-1 | 1-1 | | 13 |
| CASSQDMTDEITEAFF | MS-2 | 1.12 | 0.01 | 4-3 | 1-1 | | 14 |
| CSARDTGQGMEKLFF | MS-2 | 0.60 | 0.07 | 20-1 | 1-4 | EBV; | 15 |
| | | | | | | CD4+ | |
| CASGLRQGLTEAFF | MS-2 | 0.52 | 0.08 | 12-5 | 1-1 | EBV; | 16 |
| | | | | | | CD8+ | |
| CSGGEVEKLFF | MS-2 | 0.52 | 0.01 | 29-1 | 1-4 | | 17 |
| CASSQDRLTVYGYTF | MS-2 | 0.51 | 0.07 | 4-3 | 1-2 | | 18 |
| CASSQDFTGYTF | MS-2 | 0.46 | 0.00 | 4 | 1-2 | | 19 |
| CSVGTGGTNEKLFF | MS-2 | 0.46 | 0.04 | 29-1 | 1-4 | EBV; | 20 |
| | | | | | | CD8+ | |
| CASSQSVRLGTEAFF | MS-3 | 1.93 | 0.03 | 4-3 | 1-1 | | 21 |
| CASSQTDRGTYGYTF | MS-3 | 0.95 | 0.10 | 5-5 | 1-2 | EBV; | 22 |
| | | | | | | CD8+ | |
| CASSSSTNVNTEAFF | MS-3 | 0.84 | 0.01 | 5-1 | 1-1 | | 23 |
| CASSPLQANSPLHF | MS-3 | 0.78 | 0.06 | 6 | 1-6 | | 24 |
| CASRGGTHRGQDEAFF | MS-3 | 0.77 | 0.02 | 5-1 | 1-1 | | 25 |
| CSVIQGVIYTF | MS-3 | 0.76 | ND | 29-1 | 1-2 | | 26 |
| CASSQETGSYEQYF | MS-3 | 0.64 | 0.12 | 4-1 | 2-7 | | 27 |
| CSAREGPATNEKLFF | MS-3 | 0.63 | 0.01 | 20-1 | 1-4 | EBV / | 28 |
| | | | | | | CD4+ | |
| CASSLVGGAGTEAFF | MS-3 | 0.61 | 0.05 | 7-8 | 1-1 | | 29 |
| CASSPSRQWVVYGYTF | MS-3 | 0.58 | ND | 4-3 | 1-2 | | 30 |
| CSVEDRVYGYTF | MS-4 | 1.38 | 0.24 | 29-1 | 1-2 | | 31 |
| CSARDRDHNSPLHF | MS-4 | 0.90 | 0.25 | 20-1 | 1-6 | | 32 |
| CASRTPRGGYTF | MS-4 | 0.66 | 0.13 | 6 | 1-2 | | 33 |
| CASSPGRSNEKLFF | MS-4 | 0.54 | 0.00 | 6 | 1-4 | | 34 |
| CSALTGAAEAFF | MS-4 | 0.47 | 0.09 | 20-1 | 1-1 | | 35 |
| CSARGGGNTDTQYF | MS-4 | 0.35 | 0.01 | 20-1 | 2-3 | | 36 |
| CSAAGQGTNYGYTF | MS-4 | 0.29 | 0.02 | 20-1 | 1-2 | FLU; | 37 |
| | | | | | | CD8+ | |
| CASSQDGPRRTDTQYF | MS-4 | 0.29 | 0.22 | 14-1 | 2-3 | | 38 |
| CASRGRAATKNTEAFF | MS-4 | 0.22 | 0.04 | 13-1 | 1-1 | | 39 |
| CASSLEFSYEQYF | MS-4 | 0.20 | 0.02 | 4 | 2-7 | FLU; | 40 |
| | | | | | | CD8+ | |
| CSVYRPGGEAFF | MS-5 | 1.73 | 0.21 | 29-1 | 1-1 | EBV; | 41 |
| | | | | | | CD8+ | |
| CSASGPTGANYGYTF | MS-5 | 0.65 | 0.11 | 20-1 | 1-2 | EBV; | 42 |
| | | | | | | CD8+ | |
| CASGLDSPLSNYGYTF | MS-5 | 0.33 | 0.08 | 12-5 | 1-2 | EBV; | 43 |
| | | | | | | CD4+ | |
| CASSTTRHPSEQYF | MS-5 | 0.30 | 0.17 | 6 | 2-7 | | 44 |
| CSAKLAGGTEQFF | MS-5 | 0.29 | 0.00 | 20-1 | 2-1 | | 45 |
| CSVGTGGTNEKLFF | MS-5 | 0.25 | 0.04 | 20-1 | 1-3 | EBV; | 46 |
| | | | | | | CD8+ | |
| CSAPKQGSDTIYF | MS-5 | 0.23 | 0.01 | 29-1 | 1-4 | EBV; | 47 |
| | | | | | | CD4+ | |
| CSARGDATNEKLFF | MS-5 | 0.21 | 0.00 | 20-1 | 1-4 | | 48 |
| CSVDQTQADTQYF | MS-5 | 0.20 | 0.03 | 29-1 | 2-3 | | 49 |
| CASSPGGGITDTQYF | MS-5 | 0.19 | 0.18 | 5-6 | 2-3 | EBV; | 50 |
| | | | | | | CD8+ | |
| CASSARGTQYF | MS-6 | 0.52 | 0.04 | 6 | 2-3 | EBV; | 51 |
| | | | | | | CD4+ | |
| CASSGTYSNQPQHF | MS-6 | 0.47 | 1.41 | 12-5 | 1-5 | FLU; | 52 |
| | | | | | | CD4+ | |
| CARQGDRRGGYTF | MS-6 | 0.41 | 2.32 | 30-1 | 1-2 | | 53 |
| CASSLDRSIPLEQFF | MS-6 | 0.38 | 0.42 | 7-9 | 2 | | 54 |
| CASMEARDNEQFF | MS-6 | 0.35 | 1.89 | 5-6 | 2-1 | | 55 |
| CSVGTGLAKNIQYF | MS-6 | 0.34 | 0.07 | 29-1 | 2-4 | | 56 |
| CASSPRQGMAETQYF | MS-6 | 0.34 | 0.13 | 19-1 | 2-5 | EBV; | 57 |
| | | | | | | CD8+ | |
| CASSSPPGGWVKNIQYF | MS-6 | 0.33 | 0.03 | 7/11 | 2-4 | | 58 |
| CASSLSTVSMNTEAFF | MS-6 | 0.32 | 0.64 | 7/11 | 1-1 | | 59 |
| CAISTGGRENTEAFF | MS-6 | 0.31 | 0.02 | 10-3 | 1-1 | EBV; | 60 |
| | | | | | | CD4+ | |
| CASSLEGSGRTYEQYF | MS-7 | 1.18 | 0.86 | 11 | 2-7 | FLU; | 61 |
| | | | | | | CD8+ | |
| CASSRYESPSGNGYTF | MS-7 | 0.46 | 0.07 | 7-8 | | | 62 |
| CASSPACWGFAGLTVKVCE | MS-7 | 0.37 | 0.02 | 3/4/14 | 2-1 | | 63 |
| QFF | | | | | | | |
| CSVEAGQGSTQYF | MS-7 | 0.33 | 0.03 | 29-1 | 2-4 | | 64 |
| CASSWDVENEQFF | MS-7 | 0.30 | 0.07 | 6 | 2-1 | FLU; | 65 |
| | | | | | | CD8+ | |
| CASSPLGFSYEQYF | MS-7 | 0.28 | 0.32 | 7-6 | 2-7 | | 66 |
| CASSYSSGEVYNEQFF | MS-7 | 0.26 | 0.04 | 6 | 2-1 | FLU; | 67 |
| | | | | | | CD4+ | |
| CASSLGGVGANYGYTF | MS-7 | 0.26 | 0.04 | 7-6 | 1-2 | | 68 |
| CASARRGDNQPQHF | MS-7 | 0.25 | 0.02 | 6 | 1-5 | EBV; | 69 |
| | | | | | | CD4+ | |
| CASSGGAGLDQPQHF | MS-7 | 0.25 | 0.00 | 6 | 1-5 | | 70 |
| CASSPQGPTGTSGRAPQNIQ | MS-8 | 0.86 | 0.04 | 3/4/14 | 2-4 | | 71 |
| YF | | | | | | | |
| CASSFSAPGTDTQYF | MS-8 | 0.63 | 0.00 | 7-3 | 2-3 | | 72 |
| CASSAGRPEAFF | MS-8 | 0.62 | 0.05 | 11-2 | 1-1 | FLU; | 73 |
| | | | | | | CD8+ | |
| CASGTEAFF | MS-8 | 0.62 | 0.22 | 7/11 | 2-2 | | 74 |
| CASSLPRTDLTGELFF | MS-8 | 0.59 | 0.12 | 6 | 1-1 | | 75 |
| CASSLEQKPYEQYF | MS-8 | 0.49 | 0.08 | 7-9 | 2-7 | FLU; | 76 |
| | | | | | | CD8+ | |
| CSVDKGPGYTF | MS-8 | 0.39 | 0.07 | 29-1 | 1-2 | FLU; | 77 |
| | | | | | | CD8+ | |
| CASSLGQAYEQYF | MS-8 | 0.39 | 0.12 | 7-8 | 2-7 | | 78 |
| CASSPPLSGGIFGLTNEKLFF | MS-8 | 0.37 | 0.00 | 4-2 | 1-4 | | 79 |
| CASSTGLAGNIQYF | MS-8 | 0.31 | 0.08 | 5-1 | 2-4 | | 80 |
| CASSPLPMENTEAFF | MS-9 | 2.85 | 0.32 | 18-1 | 1-1 | EBV; | 81 |
| | | | | | | CD4+ | |
| CASSLYPHEQFF | MS-9 | 0.94 | 0.34 | 7-3 | 2-1 | | 82 |
| CASSSATGTGRFFYEKLFF | MS-9 | 0.66 | 0.14 | 12 | 1-4 | | 83 |
| CASSPPRSGTEAFF | MS-9 | 0.44 | 0.13 | 18-1 | 1-1 | FLU; | 84 |
| | | | | | | CD4+ | |
| CASSLDEREGLYNEQFF | MS-9 | 0.35 | 3.12 | 11 | 2-1 | | 85 |
| CASSLLGQGLSEKLFF | MS-9 | 0.24 | 0.04 | 5-1 | 1-4 | | 86 |
| CASSLPRVQSTPYRDSPYGY | MS-9 | 0.21 | 0.12 | 3/4/14 | 1-2 | | 87 |
| TF | | | | | | | |
| CASMGRDYTF | MS-9 | 0.20 | 0.10 | 19-1 | 1-2 | EBV; | 88 |
| | | | | | | CD8+ | |
| CASSYGLGLEAFF | MS-9 | 0.19 | 0.07 | 6 | 1-1 | EBV; | 89 |
| | | | | | | CD8+ | |
| CASSLEDIYEQYF | MS-9 | 0.19 | 0.07 | 11-2 | 2-7 | | 90 |
| CASRDAGGAETQYF | MS-10 | 0.93 | 0.32 | 6 | 2-5 | | 91 |
| CASSPLRGARRETQYF | MS-10 | 0.68 | 0.53 | 7/11 | 2-5 | | 92 |
| CASSEQGRENEQFF | MS-10 | 0.40 | 0.10 | 7-3 | 2-1 | | 93 |
| CASSEDPRGLATNEKLFF | MS-10 | 0.38 | 0.03 | 6-1 | 1-4 | | 94 |
| CASSYPTSGTDTQYF | MS-10 | 0.31 | 0.16 | 6 | 2-3 | | 95 |
| CASSQNTGELFF | MS-10 | 0.28 | 0.14 | 7-6 | 2-2 | FLU; | 96 |
| | | | | | | CD4+ | |
| CASSFDRGRVTDTQYF | MS-10 | 0.23 | 0.23 | 19-1 | 2-3 | | 97 |
| CSVENPDEKLFF | MS-10 | 0.20 | 0.00 | 29-1 | 1-4 | | 98 |
| CASSYEGGANSPLHF | MS-10 | 0.18 | 0.00 | 6 | 1-6 | | 99 |
| CASSGGTGVSGANVLTF | MS-10 | 0.17 | 0.00 | 5-1 | 2-6 | | 100 |

The ten most frequent TCRβ CDR3 sequences from the cerebrospinal fluid of patients with multiple sclerosis. The clonotype frequencies in the cerebrospinal fluid and blood are given. The last column denotes whether the same sequence was found in sorted virus reactive samples. HLA-A2 restricted Epstein-Barr virus associated public T cell receptors are in bold typeface.

TABLE 4
Reactivities of CDR3 sequences from CSF of patients and controls

| | | CSF | Blood | | | Reactivity; | SEQ ID |
|---|---|---|---|---|---|---|---|
| CDR3 | ID | (%) | (%) | V | J | phenotype | NO |
| CASSLSLGQHYGYTF | OND-1 | 0.66 | 0.13 | 7-2 | 1-1 | FLU; | 101 |
| | | | | | | CD4+ | |
| CSARQGRDHTDTQYF | OND-1 | 0.61 | 0.02 | 20-1 | 1-1 | EBV; | 102 |
| | | | | | | CD8+ | |
| CSVAPAHTFTYEQYF | OND-1 | 0.36 | 0.23 | 29-1 | 1-1 | | 103 |
| CSARGTTEAFF | OND-1 | 0.36 | 0.13 | 20-1 | 1-1 | FLU; | 104 |
| | | | | | | CD8+ | |
| CASRTTGTGEDYTF | OND-1 | 0.33 | 0.00 | 6 | 1-1 | | 105 |
| CSAISGVLSPMNEQFF | OND-1 | 0.28 | 0.01 | 20-1 | 1-1 | EBV; | 106 |
| | | | | | | CD4+ | |
| CSARPPGGGGTEAFF | OND-1 | 0.25 | ND | 20-1 | 1-1 | | 107 |
| CSAGRGTDTQYF | OND-1 | 0.23 | ND | 20-1 | 1-1 | | 108 |
| CSAIRGSVNNSPLHF | OND-1 | 0.23 | 0.01 | 20-1 | 1-1 | | 109 |
| CATMGPGTDTQYF | OND-1 | 0.22 | 0.00 | 15-1 | 1-1 | | 110 |
| CASSLDWIGNQPQHF | OND-2 | 1.44 | 0.22 | 7/11 | 1-5 | | 111 |
| CASSLDRGAPLHF | OND-2 | 1.08 | 0.05 | 6 | 1-6 | | 112 |
| CSVVPAAVYGYTF | OND-2 | 0.92 | 0.16 | 29-1 | 1-2 | EBV; | 113 |
| | | | | | | CD8+ | |
| CASSQDRSGDTQYF | OND-2 | 0.75 | 0.07 | 4 | 2-3 | | 114 |
| CSANPHTDTQYF | OND-2 | 0.72 | 0.03 | 20-1 | 2-3 | FLU; | 115 |
| | | | | | | CD8+ | |
| CASSTGGDGYTF | OND-2 | 0.59 | 0.22 | 6 | 1-2 | | 116 |
| CASSDRMNTEAFF | OND-2 | 0.57 | 0.27 | 5-1 | 1-1 | | 117 |
| CASTQGRNSPLHF | OND-2 | 0.55 | 0.00 | 6 | 1-6 | | 118 |
| CSAPGQLEKLFF | OND-2 | 0.50 | 0.00 | 6 | 2-3 | | 119 |
| CASSEGGRGDGNYGYTF | OND-2 | 0.44 | 0.16 | 20-1 | 1-2 | | 120 |
| CAISVGAGQGETQYF | OND-3 | 3.43 | 34.41 | 10-3 | 2-5 | | 121 |
| CASSLTGSRELFF | OND-3 | 0.42 | 0.05 | 7-3 | 2-2 | | 122 |
| CSVWGDGSSYEQYF | OND-3 | 0.40 | 2.47 | 29-1 | 2-7 | | 123 |
| CASSLDSSEQYF | OND-3 | 0.28 | 0.01 | 7-8 | 2-7 | EBV; | 124 |
| | | | | | | CD4+ | |
| CASSLLPSNTGELFF | OND-3 | 0.27 | 0.04 | 5-1 | 2-2 | | 125 |
| CASSFRPGQGAGGANVLTF | OND-3 | 0.25 | 0.02 | 12 | 2-6 | | 126 |
| CASTVYRGYNTEAFF | OND-3 | 0.23 | 0.45 | 5-6 | 1-1 | | 127 |
| CASSLWISGLGEQFF | OND-3 | 0.19 | 0.01 | 7-2 | 2 | | 128 |
| CASSSGTGQGETQYF | OND-3 | 0.19 | 2.93 | 11-2 | 2-5 | | 129 |
| CASSDRTHDTQYF | OND-3 | 0.17 | 0.06 | 10-1 | 2-3 | | 130 |
| CASSHRTENTEAFF | OND-4 | 0.83 | 1.13 | 4-1 | 1-1 | | 131 |
| CASSYMGRASNTEAFF | OND-4 | 0.22 | 0.06 | 6 | 1-1 | EBV; | 132 |
| | | | | | | CD4+ | |
| CASNDRGLDGEQYF | OND-4 | 0.20 | 0.04 | 6 | 2-7 | EBV; | 133 |
| | | | | | | CD4+ | |
| CASSPRQGPTGELFF | OND-4 | 0.18 | 0.06 | 7-8 | 2-2 | EBV; | 134 |
| | | | | | | CD8+ | |
| CASSWTGTYQETQYF | OND-4 | 0.17 | 0.09 | 5-6 | 2-5 | EBV; | 135 |
| | | | | | | CD8+ | |
| CASSLQLAGGLETQYF | OND-4 | 0.15 | 0.00 | 7/11 | 2-5 | | 136 |
| CSVEDLGGVDTQYF | OND-4 | 0.13 | 0.01 | 29-1 | 2-3 | | 137 |
| CASSLEGNEQFF | OND-4 | 0.13 | 0.02 | 12 | 2-1 | EBV; | 138 |
| | | | | | | CD8+ | |
| CASRTKGGMNTEAFF | OND-4 | 0.13 | 0.04 | 6 | 1-1 | EBV; | 139 |
| | | | | | | CD8+ | |
| CASSRQGAYGAGGRYNEQF | OND-4 | 0.11 | ND | 4-2 | 2-1 | | 140 |
| F | | | | | | | |
| CSVDPGTGELFF | OND-5 | 4.31 | 11.83 | 29-1 | 2-2 | | 141 |
| CSVGSGEGYEQYF | OND-5 | 1.45 | 2.92 | 29-1 | 2-7 | | 142 |
| CSVDSDYEQYF | OND-5 | 1.28 | 0.66 | 29-1 | 2-7 | | 143 |
| CASSDSGGVDYGYTF | OND-5 | 1.13 | 0.28 | 6 | 1-2 | | 144 |
| CSATGANSYEQYF | OND-5 | 0.92 | 6.25 | 29-1 | 2-7 | | 145 |
| CSVGGSFYGYTF | OND-5 | 0.81 | 1.33 | 29-1 | 1-2 | | 146 |
| CASSDPAGVFRQPQHF | OND-5 | 0.71 | 0.07 | 6 | 1-5 | EBV; | 147 |
| | | | | | | CD4+ | |
| CASSLETTSSNTGELFF | OND-5 | 0.53 | 0.23 | 11 | 2-2 | EBV; | 148 |
| | | | | | | CD8+ | |
| CASSQDPPDTQYF | OND-5 | 0.44 | 0.06 | 14-1 | 2-3 | | 149 |
| CASSSPGGANYEQFF | OND-5 | 0.38 | 0.25 | 5-5 | 2-1 | EBV; | 150 |
| | | | | | | CD8+ | |
| CASSPPPGVKETQYF | OND-6 | 0.96 | 0.12 | 14-1 | 2-5 | | 151 |
| CSARSLRTVHNEKLFF | OND-6 | 0.44 | 0.06 | 20-1 | 1-4 | | 152 |
| CASIWDRTPPTGVNGYTF | OND-6 | 0.25 | 0.02 | 5 | 1-2 | EBV; | 153 |
| | | | | | | CD8+ | |
| CASSSYGQETQYF | OND-6 | 0.24 | 0.09 | 7-3 | 2-5 | EBV; | 154 |
| | | | | | | CD8+ | |
| CASSEWTAPNQPQHF | OND-6 | 0.20 | ND | 6-1 | 1-5 | | 155 |
| CASSPTSNLGTQYF | OND-6 | 0.19 | 0.01 | 6 | 2-5 | | 156 |
| CSVVAGIYGYTF | OND-6 | 0.17 | 0.00 | 29-1 | 1-2 | | 157 |
| CASTRGDGTEAFF | OND-6 | 0.17 | 0.01 | 7-9 | 1-1 | EBV; | 158 |
| | | | | | | CD4+ | |
| CASSQETALNSPLHF | OND-6 | 0.14 | 0.07 | 4-1 | 1-6 | FLU;CD8+ | 159 |
| CASSLVKRGAYNEQFF | OND-6 | 0.14 | 0.03 | 7-8 | 2-1 | EBV; | 160 |
| | | | | | | CD8+ | |
| CASSSVGGDTQYF | OND-7 | 2.86 | 0.48 | 18-1 | 2-3 | | 161 |
| CASSIPRQGSGYTF | OND-7 | 1.76 | 0.08 | 19-1 | 1-2 | | 162 |
| CASSIEGQKNIQYF | OND-7 | 1.73 | 0.49 | 19-1 | 2-4 | FLU; | 163 |
| | | | | | | CD4+ | |
| CASSPSAVGTDTQYF | OND-7 | 1.66 | 0.40 | 6 | 2-3 | EBV; | 164 |
| | | | | | | CD4+ | |
| CASSPVDPMGQPQHF | OND-7 | 1.04 | 0.19 | 18-1 | 1-5 | | 165 |
| CASSITSGSYNEQFF | OND-7 | 0.93 | 1.63 | 19-1 | 2-1 | | 166 |
| CASSTTGNTEAFF | OND-7 | 0.68 | 0.08 | 6 | 1-1 | | 167 |
| CAGRGTGQRMGYNSPLHF | OND-7 | 0.58 | 0.06 | 6/10 | 1-6 | | 168 |
| CASSLEATGAYNEQFF | OND-7 | 0.56 | 0.12 | 11 | 2-1 | | 169 |
| CASSFGGGNTDTQYF | OND-7 | 0.55 | 0.34 | 5-1 | 2-3 | | 170 |
| CASSYWAAGELFF | OND-8 | 1.47 | 1.53 | 6 | 2-2 | EBV; | 171 |
| | | | | | | CD8+ | |
| CASSQSMGRTNTGELFF | OND-8 | 1.34 | 1.18 | 23-1 | 2-2 | | 172 |
| CASSYLAGDIQYF | OND-8 | 1.02 | 0.66 | 6 | 2-4 | EBV; | 173 |
| | | | | | | CD8+ | |
| CASSYFSSSYEQYF | OND-8 | 1.01 | 0.03 | 6 | 2-7 | | 174 |
| CASSLQALNTEAFF | OND-8 | 0.59 | 0.19 | 7-2 | 1-1 | | 175 |
| CASSLAEDSYEQYF | OND-8 | 0.45 | 0.62 | 7-8 | 2-7 | | 176 |
| CASSDSGGSNNEQFF | OND-8 | 0.45 | 0.30 | 2-1 | 2-1 | | 177 |
| CASRKQGGNYGYTF | OND-8 | 0.44 | 0.29 | 6 | 1-2 | | 178 |
| CASSRQPGQGAGGANVLTF | OND-8 | 0.30 | 0.09 | 6/12 | 2-6 | | 179 |
| CASSYSPGLAGDEQYF | OND-8 | 0.26 | 0.10 | 6 | 1-4 | FLU; | 180 |
| | | | | | | CD4+ | |
| CAISESGRSTDTQYF | OND-9 | 0.67 | 0.09 | 10-3 | 2-3 | FLU; | 181 |
| | | | | | | CD4+ | |
| CASSLTYSYEQYF | OND-9 | 0.47 | 2.93 | 7-2 | 2-7 | | 182 |
| CASRGEQGFRQPQHF | OND-9 | 0.46 | 0.02 | 6 | 1-5 | | 183 |
| CSVGTSGTQYF | OND-9 | 0.36 | ND | 29-1 | 2-3 | | 184 |
| CASSRLQGNTQYF | OND-9 | 0.32 | 0.01 | 18-1 | 2-3 | EBV; | 185 |
| | | | | | | CD4+ | |
| CASSFREKYEQYF | OND-9 | 0.31 | 0.29 | 7-9 | 2-7 | EBV; | 186 |
| | | | | | | CD8+ | |
| CASTLAGGHEKLFF | OND-9 | 0.29 | 0.01 | 6 | 1-4 | EBV; | 187 |
| | | | | | | CD4+ | |
| CAIPAGSMDTGELFF | OND-9 | 0.24 | 0.01 | 10-3 | 2-2 | EBV; | 188 |
| | | | | | | CD4+ | |
| CASSLLGGTLSTGELFF | OND-9 | 0.21 | 0.00 | 7-3 | 2-2 | | 189 |
| CASSFFFIPRTGGNYGYTF | OND-9 | 0.21 | 1.47 | 7/11 | 1-2 | FLU; | 190 |
| | | | | | | CD4+ | |
| CASSLGTGWAGELFF | OND-10 | 1.63 | 0.33 | 7-8 | 2-2 | EBV; | 191 |
| | | | | | | CD8+ | |
| CASSPDRVRETQYF | OND-10 | 1.14 | 0.00 | 6 | 2-5 | | 192 |
| CASSFWGAGGREKLFF | OND-10 | 0.73 | 0.03 | 7-8 | 1-4 | | 193 |
| CASSGISGNPYEQYF | OND-10 | 0.67 | 0.01 | 6-1 | 2-7 | | 194 |
| CASSLGDTEAFF | OND-10 | 0.37 | 0.22 | 7-9 | 1-1 | | 195 |
| CASSARDTFTGELFF | OND-10 | 0.36 | 0.15 | 6 | 2-2 | EBV; | 196 |
| | | | | | | CD8+ | |
| CASSPLPSEQYF | OND-10 | 0.35 | 0.07 | 18-1 | 2-7 | EBV; | 197 |
| | | | | | | CD4+ | |
| CASSPWGSNEQFF | OND-10 | 0.35 | 0.03 | 18-1 | 2-1 | | 198 |
| CASGRTGSYNEQFF | OND-10 | 0.30 | 0.04 | 6 | 2-1 | | 199 |
| CAWSKLGPPDTQYF | OND-10 | 0.26 | 0.02 | 30-1 | 2-3 | | 200 |
| CSARDQTSGSSYPETQYF | OND-11 | 3.01 | 0.00 | 20-1 | 2-5 | | 201 |
| CASSYLGRDTQYF | OND-11 | 2.63 | 0.00 | 6 | 2-3 | | 202 |
| CASSLGDTEAFF | OND-11 | 2.34 | 0.00 | 7-9 | 1-1 | | 203 |
| CASGPPGGADGYTF | OND-11 | 1.77 | 0.00 | 12-5 | 1-2 | | 204 |
| CASSLVRGRAGELFF | OND-11 | 1.54 | ND | 4-1 | 2-2 | | 205 |
| CASSPTGLSRGAFF | OND-11 | 1.35 | ND | 14-1 | 1-1 | | 206 |
| CASSKNTGIRTDTQYF | OND-11 | 1.34 | ND | 7-8 | 2-3 | | 207 |
| CARSGTGIVNEQFF | OND-11 | 1.27 | 0.00 | 7-2 | 2-1 | | 208 |
| CASSQDRREAFF | OND-11 | 1.06 | ND | 6 | 1-1 | | 209 |
| CASSHQILPGTGPGNTIYF | OND-11 | 0.97 | 0.00 | 14-1 | 1-3 | | 210 |

The ten most frequent TCRβ CDR3 sequences from the cerebrospinal fluid of controls with other neuroinflammatory diseases. The clonotype frequencies in the cerebrospinal fluid and blood are given. The last column denotes whether the same sequence was found in sorted virus reactive samples.

### Example 4

### Public EBV-Specific TCRs are Enriched in the CSF of Patients with MS, but not OND

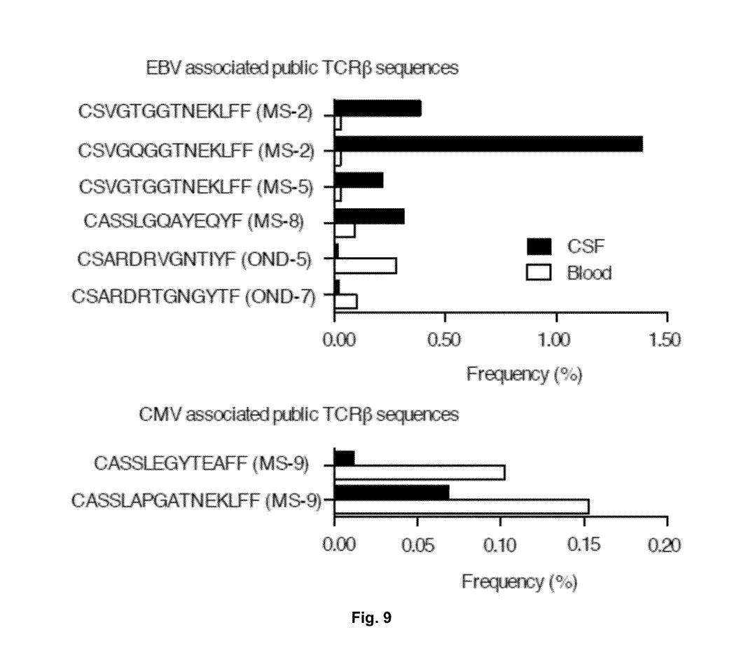

During the development of embodiments of the technology provided herein, data were collected indicating that a total of 671 CDR3 sequences from CSF and 330 from blood of 10 MS patients were present at copy numbers above 0.1% of the total repertoire and were thus regarded as expanded T cell clones. Among these, MS-2 and MS-5 shared one sequence (CSVGTGGTNEKLFF; SEQ ID NO:20) with identical V- and J-genes. This sequence has previously been identified as an HLA-A2-restricted EBV-specific public TCR (Venturi et al., supra). The expanded T cell clones from either blood or CSF of patients with MS and OND identified by these experiments were compared with data from previous studies that identified TCRs associated with public CD8+ T cell responses against EBV, CMV, Influenza A virus, and Herpes Simplex Virus-2 (Table 5; FIG. 9). In addition to the already described EBV-associated sequence, another HLA-A2-restricted EBV-specific public TCR was identified (CSVGQGGTNEKLFF) among the CDR3 sequences from MS-2, and an HLA-B8-restricted EBV-specific public TCR(CASSLGQAYEQYF) in MS-8 (FIG. 9). Two HLA-A2-restricted EBV-associated public TCRs were identified among CDR3 sequences from OND-5 and OND-7. All four EBV-associated public TCRs from the MS patients were enriched in the CSF, whereas those from the controls with OND were more frequent in blood. Further, the two CMV-associated public TCRs identified in MS-9 were more prevalent in blood (FIG. 9).

The TCRβ CDR3 sequences of expanded T cell clones were compared with those previously described in the non-redundant protein database of the National Center for Biotechnology Information (NCBI) and in papers identified at the NCBI PubMed database using “multiple sclerosis” and “T cell receptor” as MeSH major topics. This revealed that one of the identified HLA-A2 restricted EBV-specific TCRs (CSVGTGGTNEKLFF; SEQ ID NO:20), and a close match (CSVGSGGTNEKLFF; SEQ ID NO:254), were listed among a few monoclonal expansions obtained from several MS lesions in brains from two out of three investigated HLA-A2 positive MS patients, although this was not recognized by the authors (Junker A, et al., Brain. 2007; 130(pt 11):2789-2799).

TABLE 5
Previously identified public T cell receptors associated with CD8+ T cell
responses against human viruses

| | | | HLA | | | SEQ ID |
|---|---|---|---|---|---|---|
| TCRβ CDR3 sequence | V | J | restriction | Specificity | References | NO |
| CSARDGTGNGYT | 20-1 | 1-2 | A*0201 | EBV | (1) | 211 |
| CSARDRTGNGYT | 20-1 | 1-2 | A*0201 | EBV | (1) | 212 |
| CSARDRVGNTIY | 20-1 | 1-3 | A*0201 | EBV | (1) | 213 |
| CSARVGVGNTIY | 20-1 | 1-3 | A*0201 | EBV | (1) | 214 |
| CSVGTGGTNEKLF | 29-1 | 1-4 | A*0201 | EBV | (1) | 215 |
| CSSQEGGYGYT | 29-1 | 1-2 | A*0201 | EBV | (1) | 216 |
| CSARDQTGNGYT | 20-1 | 1-2 | A*0201 | EBV | (1) | 217 |
| CSARDRIGNGYT | 20-1 | 1-2 | A*0201 | EBV | (1) | 218 |
| CSARIGVGNTIY | 20-1 | 1-3 | A*0201 | EBV | (1) | 219 |
| CSARSGVGNTIY | 20-1 | 1-3 | A*0201 | EBV | (1) | 220 |
| CASSEGRVSPGELF | 2 | 2-2 | A*0201 | EBV | (1) | 221 |
| CSVGQGGTNEKLF | 29-1 | 1-4 | A*0201 | EBV | (2) | 222 |
| CASSLGQAYEQYF | 7-8 | 2-7 | B*0801 | EBV | (3) | 223 |
| CAISTGDSNQPQHF | 10-3 | 1-5 | B*3501 | EBV | (4) | 224 |
| CAIGTGDSNQPQHF | 10-3 | 1-5 | B*3501 | EBV | (4) | 225 |
| CATSTGDSNQPQHF | 10-3 | 1-5 | B*3501 | EBV | (4) | 226 |
| CASSARSGELFF | 9 | 2-2 | B*3501/08 | EBV | (5) | 227 |
| CASSARTGELFF | 9 | 2-2 | B*3501 | EBV | (5) | 228 |
| CASSVRTGELFF | 9 | 2-2 | B*3501 | EBV | (5) | 229 |
| CASRYRDDSYNEQFF | 7-9 | 2-1 | B*4405 | EBV | (4, 6) | 230 |
| CASSSRSSYEQYF | 19 | 2-7 | A*0201 | Influenza A | (7) | 231 |
| CASSIRSSYEQYF | 19 | 2-7 | A*0201 | Influenza A | (7) | 232 |
| CASSMRSSYEQYF | 19 | 2-7 | A*0201 | Influenza A | (7) | 233 |
| CASSTRSTDTQYF | 19 | 2-3 | A*0201 | Influenza A | (7) | 234 |
| CASSMRSTDTQYF | 19 | 2-3 | A*0201 | Influenza A | (7) | 235 |
| CASSIRSAYEQYF | 19 | 2-7 | A*0201 | Influenza A | (7) | 236 |
| CASSTGSYGYTF | 19 | 1-2 | A*0201 | Influenza A | (7) | 237 |
| CASSIGNYGYTF | 19 | 1-2 | A*0201 | Influenza A | (7) | 238 |
| CASSSANYGYT | 12-4 | 1-2 | A*0201 | CMV | (1) | 239 |
| CASSLAPGATNEKLF | 7-6 | 1-4 | A*0201 | CMV | (1) | 240 |
| CASSSAHYGYT | 12-4 | 1-2 | A*0201 | CMV | (1) | 241 |
| CASSLEGYTEAF | 27 | 1-1 | A*0201 | CMV | (1) | 242 |
| CASSSAYYGYT | 12-4 | 1-2 | A*0201 | CMV | (1) | 243 |
| CASSLVGGRYGYT | 12-4 | 1-2 | A*0201 | CMV | (1) | 244 |
| CASSVVNEQF | 12-4 | 2-1 | A*0201 | CMV | (1) | 245 |
| CASSLTSGSPYNEQF | 27 | 1-1 | A*0201 | CMV | (1) | 246 |
| CASSFQGYTEAF | 28 | 1-1 | A*0201 | CMV | (1) | 247 |
| CASRPGTSSYEQYF | 28 | 2-7 | B*3508 | CMV | (8) | 248 |
| CASSPQTGTGGYGYTFG | 13-1 | 1-2 | B*0702 | CMV | (9) | 249 |
| CASSVWGTDTQYF | 9 | 2-3 | B*0702 | HSV | (10) | 250 |
| CASRRQGRDNEQFF | 12-4 | 2-1 | B*0702 | HSV | (10) | 251 |
| CATRIGWGTDTQYF | 5-1 | 2-3 | B*0702 | HSV | (10) | 252 |

EBV, Epstein-Barr virus; CMV, cytomegalovirus; HSV, Herpes simplex virus

### REFERENCES IN TABLE 5

1. Venturi, V., Chin, H. Y., Asher, T. E., Ladell, K., Scheinberg, P., Bornstein, E., van Bockel, D., Kelleher, A. D., Douek, D. C., Price, D. A., et al. 2008. TCR beta-chain sharing in human CD8+ T cell responses to cytomegalovirus and EBV. J Immunol 181:7853-7862.
2. Miconnet, I., Marrau, A., Farina, A., Taffe, P., Vigano, S., Harari, A., and Pantaleo, G. 2011. Large TCR diversity of virus-specific CD8 T cells provides the mechanistic basis for massive TCR renewal after antigen exposure. J Immunol 186:7039-7049.
3. Argaet, V. P., Schmidt, C. W., Burrows, S. R., Silins, S. L., Kurilla, M. G., Doolan, D. L., Suhrbier, A., Moss, D. J., Kieff, E., Sculley, T. B., et al. 1994. Dominant selection of an invariant T cell antigen receptor in response to persistent infection by Epstein-Barr virus. Exp Med 180:2335-2340.
4. Miles, J. J., Silins, S. L., Brooks, A. G., Davis, J. E., Misko, I., and Burrows, S. R. 2005. T-cell grit: large clonal expansions of virus-specific CD8+ T cells can dominate in the peripheral circulation for at least 18 years. Blood 106:4412-4413.
5. Miles, J. J., Borg, N. A., Brennan, R. M., Tynan, F. E., Kjer-Nielsen, L., Silins, S. L., Bell, M. J., Burrows, J. M., McCluskey, J., Rossjohn, J., et al. 2006. TCR alpha genes direct MHC restriction in the potent human T cell response to a class I-bound viral epitope. J Immunol 177:6804-6814.
6. Neller, M. A., Burrows, J. M., Rist, M. J., Miles, J. J., and Burrows, S. R. 2013. High frequency of herpesvirus-specific clonotypes in the human T cell repertoire can remain stable over decades with minimal turnover. J Virol 87:697-700.
7. Lehner, P. J., Wang, E. C., Moss, P. A., Williams, S., Platt, K., Friedman, S. M., Bell, J. I., and Borysiewicz, L. K. 1995. Human HLA-A0201-restricted cytotoxic T lymphocyte recognition of influenza A is dominated by T cells bearing the V beta 17 gene segment. J Exp Med 181:79-91.
8. Wynn, K. K., Fulton, Z., Cooper, L., Silins, S. L., Gras, S., Archbold, J. K., Tynan, F. E., Miles, J. J., McCluskey, J., Burrows, S. R., et al. 2008. Impact of clonal competition for peptide-MHC complexes on the CD8+ T-cell repertoire selection in a persistent viral infection. Blood 111:4283-4292.
9. Khan, N., Shariff, N., Cobbold, M., Bruton, R., Ainsworth, J. A., Sinclair, A. J., Nayak, L., and Moss, P. A. 2002. Cytomegalovirus seropositivity drives the CD8 T cell repertoire toward greater clonality in healthy elderly individuals. J Immunol 169:1984-1992.
10. Dong, L., Li, P., Oenema, T., McClurkan, C. L., and Koelle, D. M. 2010. Public TCR use by herpes simplex virus-2-specific human CD8 CTLs. J Immunol 184:3063-3071.

All publications and patents mentioned in the above specification are herein incorporated by reference in their entirety for all purposes. Various modifications and variations of the described compositions, methods, and uses of the technology will be apparent to those skilled in the art without departing from the scope and spirit of the technology as described. Although the technology has been described in connection with specific exemplary embodiments, it should be understood that the invention as claimed should not be unduly limited to such specific embodiments. Indeed, various modifications of the described modes for carrying out the invention that are obvious to those skilled in the art are intended to be within the scope of the following claims.

We claim:

1. A method for identifying a disease-related antigen, the method comprising:
a) sequencing a set of T cell receptor genes from a disease-related sample from a subject having a disease;
b) sequencing a set of T cell receptor genes from a control sample;
c) comparing the T cell receptor gene frequencies of the set of T cell receptor genes from the disease-related sample with the T cell receptor gene frequencies of the set of T cell receptor genes from the control sample to identify T cell receptor genes enriched in the disease-related sample,
wherein the T cell receptor genes enriched in the disease-related sample identify a disease-related antigen associated with the disease.

2. The method of claim 1 wherein the disease is an autoimmune or inflammatory disease.

3. The method of claim 1 wherein the T cell genes encode T cell receptor alpha-chain polypeptides and T cell receptor beta-chain polypeptides.

4. The method of claim 1 wherein the T cell genes encode T cell receptor alpha-chain polypeptides or T cell receptor beta-chain polypeptides.

5. The method of claim 1 wherein the T cell genes encode the T cell receptor beta chain complementarity determining regions.

6. The method of claim 1 wherein the T cell genes encode T cell receptor beta chain complementarity determining region 3 (CDR3).

7. The method of claim 1 wherein the disease-related sample is selected from the group consisting of a tissue sample from the central nervous system, a sample from a disease affected organ, cerebrospinal fluid and synovial fluid.

8. The method of claim 1 wherein the control sample is a blood sample from the subject.

9. The method of claim 1 further comprising collecting cell samples from the subject having the disease to provide the disease-related sample.

10. The method of claim 1 further comprising collecting cell samples from an organ or tissue from the subject having the disease to provide the disease-related sample.

11. The method of claim 1 wherein the control sample is a blood sample and the method further comprises stimulating the blood sample with a test antigen and a control antigen.

12. The method of claim 11 further comprising isolating T cells that respond to the test antigen and isolating T cells that respond to the control antigen to provide antigen-responding samples.

13. The method of claim 12 wherein a disease-related antigen is identified by high frequencies of the same TCR genes in the disease-related sample and an antigen-responding sample.

14. A method of identifying a disease-related antigen, the method comprising:
a) contacting a control sample from a subject diagnosed with an inflammatory or an autoimmune disease with a test antigen and a control antigen;
b) isolating T cells from the control sample that respond to said test antigen to provide control sample test-antigen responsive T cells and isolating T cells from the control sample that respond to said control antigen to provide control sample control-antigen responsive T cells;
c) sequencing T-cell receptor genes from said control sample test-antigen responsive T cells to provide control sample test-antigen responsive gene sequences and sequencing T-cell receptor genes from said control sample control-antigen responsive T cells to provide control sample control-antigen responsive gene sequences;
d) isolating T cells from the disease-related sample to provide disease-related T cells;
e) sequencing T-cell receptor genes from said disease-related T cells to provide disease related gene sequences; and
f) comparing the frequency of said control sample test-antigen responsive gene sequences and control sample control-antigen responsive gene sequences with the frequency of said disease-related gene sequences,
wherein the gene sequences enriched in the disease-related sample identify a disease-related antigen associated with the disease.

15. The method of claim 14, wherein the control sample is a peripheral blood sample.

16. The method of claim 14 wherein the T cells are CD4+ or CD8+ T cells.

17. The method of claim 14 wherein the disease is an autoimmune or inflammatory disease.

18. The method of claim 14 further comprising contacting a control sample from a subject not diagnosed with an inflammatory or an autoimmune disease with the test antigen and the control antigen.

19. The method of claim 14, further comprising repeating step (a) by contacting a plurality of control samples with a plurality of test antigens and sequencing to provide a reference library for comparison with disease-related gene sequences.

20. A method of treating a disease comprising an aberrant immunological response to a disease-related antigen, the method comprising identifying the disease-related antigen according to the method of claim 1 and minimizing the immunological response to the disease-related antigen in a patient.


SEQUENCE LISTING (254 sequences)

SEQ ID NO: 1 - protein, 16 aa, Homo sapiens
CASSQDRAGGPYGYTF

SEQ ID NO: 2 - protein, 11 aa, Homo sapiens
CASSLGRGYTF

SEQ ID NO: 3 - protein, 14 aa, Homo sapiens
CASTWGVVAGELFF

SEQ ID NO: 4 - protein, 15 aa, Homo sapiens
CASSPHGGSANVLTF

SEQ ID NO: 5 - protein, 13 aa, Homo sapiens
CASSPGTDYGYTF

SEQ ID NO: 6 - protein, 16 aa, Homo sapiens
CASSQHTGIPGNTIYF

SEQ ID NO: 7 - protein, 11 aa, Homo sapiens
CSARAERGQHF

SEQ ID NO: 8 - protein, 13 aa, Homo sapiens
CASSLAYTGELFF

SEQ ID NO: 9 - protein, 15 aa, Homo sapiens
CASSPTQGTFYGYTF

SEQ ID NO: 10 - protein, 14 aa, Homo sapiens
CASTSSPLSTEAFF

SEQ ID NO: 11 - protein, 15 aa, Homo sapiens
CSTPRDNAKNTEAFF

SEQ ID NO: 12 - protein, 14 aa, Homo sapiens
CSVGQGGTNEKLFF

SEQ ID NO: 13 - protein, 13 aa, Homo sapiens
CSGLKQGGTEAFF

SEQ ID NO: 14 - protein, 16 aa, Homo sapiens
CASSQDMTDEITEAFF

SEQ ID NO: 15 - protein, 15 aa, Homo sapiens
CSARDTGQGMEKLFF

SEQ ID NO: 16 - protein, 14 aa, Homo sapiens
CASGLRQGLTEAFF

SEQ ID NO: 17 - protein, 11 aa, Homo sapiens
CSGGEVEKLFF

SEQ ID NO: 18 - protein, 15 aa, Homo sapiens
CASSQDRLTVYGYTF

SEQ ID NO: 19 - protein, 12 aa, Homo sapiens
CASSQDFTGYTF

SEQ ID NO: 20 - protein, 14 aa, Homo sapiens
CSVGTGGTNEKLFF

SEQ ID NO: 21 - protein, 15 aa, Homo sapiens
CASSQSVRLGTEAFF

SEQ ID NO: 22 - protein, 15 aa, Homo sapiens
CASSQTDRGTYGYTF

SEQ ID NO: 23 - protein, 15 aa, Homo sapiens
CASSSSTNVNTEAFF

SEQ ID NO: 24 - protein, 14 aa, Homo sapiens
CASSPLQANSPLHF

SEQ ID NO: 25 - protein, 16 aa, Homo sapiens
CASRGGTHRGQDEAFF

SEQ ID NO: 26 - protein, 11 aa, Homo sapiens
CSVIQGVIYTF

SEQ ID NO: 27 - protein, 14 aa, Homo sapiens
CASSQETGSYEQYF

SEQ ID NO: 28 - protein, 15 aa, Homo sapiens
CSAREGPATNEKLFF

SEQ ID NO: 29 - protein, 15 aa, Homo sapiens
CASSLVGGAGTEAFF

SEQ ID NO: 30 - protein, 16 aa, Homo sapiens
CASSPSRQWVVYGYTF

SEQ ID NO: 31 - protein, 12 aa, Homo sapiens
CSVEDRVYGYTF

SEQ ID NO: 32 - protein, 14 aa, Homo sapiens
CSARDRDHNSPLHF

SEQ ID NO: 33 - protein, 12 aa, Homo sapiens
CASRTPRGGYTF

SEQ ID NO: 34 - protein, 14 aa, Homo sapiens
CASSPGRSNEKLFF

SEQ ID NO: 35 - protein, 12 aa, Homo sapiens
CSALTGAAEAFF

SEQ ID NO: 36 - protein, 14 aa, Homo sapiens
CSARGGGNTDTQYF

SEQ ID NO: 37 - protein, 14 aa, Homo sapiens
CSAAGQGTNYGYTF

SEQ ID NO: 38 - protein, 16 aa, Homo sapiens
CASSQDGPRRTDTQYF

SEQ ID NO: 39 - protein, 16 aa, Homo sapiens
CASRGRAATKNTEAFF

SEQ ID NO: 40 - protein, 13 aa, Homo sapiens
CASSLEFSYEQYF

SEQ ID NO: 41 - protein, 12 aa, Homo sapiens
CSVYRPGGEAFF

SEQ ID NO: 42 - protein, 15 aa, Homo sapiens
CSASGPTGANYGYTF

SEQ ID NO: 43 - protein, 16 aa, Homo sapiens
CASGLDSPLSNYGYTF

SEQ ID NO: 44 - protein, 14 aa, Homo sapiens
CASSTTRHPSEQYF

SEQ ID NO: 45 - protein, 13 aa, Homo sapiens
CSAKLAGGTEQFF

SEQ ID NO: 46 - protein, 14 aa, Homo sapiens
CSVGTGGTNEKLFF

SEQ ID NO: 47 - protein, 13 aa, Homo sapiens
CSAPKQGSDTIYF

SEQ ID NO: 48 - protein, 14 aa, Homo sapiens
CSARGDATNEKLFF

SEQ ID NO: 49 - protein, 13 aa, Homo sapiens
CSVDQTQADTQYF

SEQ ID NO: 50 - protein, 15 aa, Homo sapiens
CASSPGGGITDTQYF

SEQ ID NO: 51 - protein, 11 aa, Homo sapiens
CASSARGTQYF

SEQ ID NO: 52 - protein, 14 aa, Homo sapiens
CASSGTYSNQPQHF

SEQ ID NO: 53 - protein, 13 aa, Homo sapiens
CARQGDRRGGYTF

SEQ ID NO: 54 - protein, 15 aa, Homo sapiens
CASSLDRSIPLEQFF

SEQ ID NO: 55 - protein, 13 aa, Homo sapiens
CASMEARDNEQFF

SEQ ID NO: 56 - protein, 14 aa, Homo sapiens
CSVGTGLAKNIQYF

SEQ ID NO: 57 - protein, 15 aa, Homo sapiens
CASSPRQGMAETQYF

SEQ ID NO: 58 - protein, 17 aa, Homo sapiens
CASSSPPGGWVKNIQYF

SEQ ID NO: 59 - protein, 16 aa, Homo sapiens
CASSLSTVSMNTEAFF

SEQ ID NO: 60 - protein, 15 aa, Homo sapiens
CAISTGGRENTEAFF

SEQ ID NO: 61 - protein, 16 aa, Homo sapiens
CASSLEGSGRTYEQYF

SEQ ID NO: 62 - protein, 16 aa, Homo sapiens
CASSRYESPSGNGYTF

SEQ ID NO: 63 - protein, 22 aa, Homo sapiens
CASSPACWGFAGLTVKVCEQFF

SEQ ID NO: 64 - protein, 13 aa, Homo sapiens
CSVEAGQGSTQYF

SEQ ID NO: 65 - protein, 13 aa, Homo sapiens
CASSWDVENEQFF

SEQ ID NO: 66 - protein, 14 aa, Homo sapiens
CASSPLGFSYEQYF

SEQ ID NO: 67 - protein, 16 aa, Homo sapiens
CASSYSSGEVYNEQFF

SEQ ID NO: 68 - protein, 16 aa, Homo sapiens
CASSLGGVGANYGYTF

SEQ ID NO: 69 - protein, 14 aa, Homo sapiens
CASARRGDNQPQHF

SEQ ID NO: 70 - protein, 15 aa, Homo sapiens
CASSGGAGLDQPQHF

SEQ ID NO: 71 - protein, 22 aa, Homo sapiens
CASSPQGPTGTSGRAPQNIQYF

SEQ ID NO: 72 - protein, 15 aa, Homo sapiens
CASSFSAPGTDTQYF

SEQ ID NO: 73 - protein, 12 aa, Homo sapiens
CASSAGRPEAFF

SEQ ID NO: 74 - protein, 9 aa, Homo sapiens
CASGTEAFF

SEQ ID NO: 75 - protein, 16 aa, Homo sapiens
CASSLPRTDLTGELFF

SEQ ID NO: 76 - protein, 14 aa, Homo sapiens
CASSLEQKPYEQYF

SEQ ID NO: 77 - protein, 11 aa, Homo sapiens
CSVDKGPGYTF

SEQ ID NO: 78 - protein, 13 aa, Homo sapiens
CASSLGQAYEQYF

SEQ ID NO: 79 - protein, 21 aa, Homo sapiens
CASSPPLSGGIFGLTNEKLFF

SEQ ID NO: 80 - protein, 14 aa, Homo sapiens
CASSTGLAGNIQYF

SEQ ID NO: 81 - protein, 15 aa, Homo sapiens
CASSPLPMENTEAFF

SEQ ID NO: 82 - protein, 12 aa, Homo sapiens
CASSLYPHEQFF

SEQ ID NO: 83 - protein, 19 aa, Homo sapiens
CASSSATGTGRFFYEKLFF

SEQ ID NO: 84 - protein, 14 aa, Homo sapiens
CASSPPRSGTEAFF

SEQ ID NO: 85 - protein, 17 aa, Homo sapiens
CASSLDEREGLYNEQFF

SEQ ID NO: 86 - protein, 16 aa, Homo sapiens
CASSLLGQGLSEKLFF

SEQ ID NO: 87 - protein, 22 aa, Homo sapiens
CASSLPRVQSTPYRDSPYGYTF

SEQ ID NO: 88 - protein, 10 aa, Homo sapiens
CASMGRDYTF

SEQ ID NO: 89 - protein, 13 aa, Homo sapiens
CASSYGLGLEAFF

SEQ ID NO: 90 - protein, 13 aa, Homo sapiens
CASSLEDIYEQYF

SEQ ID NO: 91 - protein, 14 aa, Homo sapiens
CASRDAGGAETQYF

SEQ ID NO: 92 - protein, 16 aa, Homo sapiens
CASSPLRGARRETQYF

SEQ ID NO: 93 - protein, 14 aa, Homo sapiens
CASSEQGRENEQFF

SEQ ID NO: 94 - protein, 18 aa, Homo sapiens
CASSEDPRGLATNEKLFF

SEQ ID NO: 95 - protein, 15 aa, Homo sapiens
CASSYPTSGTDTQYF

SEQ ID NO: 96 - protein, 12 aa, Homo sapiens
CASSQNTGELFF

SEQ ID NO: 97 - protein, 16 aa, Homo sapiens
CASSFDRGRVTDTQYF

SEQ ID NO: 98 - protein, 12 aa, Homo sapiens
CSVENPDEKLFF

SEQ ID NO: 99 - protein, 15 aa, Homo sapiens
CASSYEGGANSPLHF

SEQ ID NO: 100 - protein, 17 aa, Homo sapiens
CASSGGTGVSGANVLTF

SEQ ID NO: 101 - protein, 15 aa, Homo sapiens
CASSLSLGQHYGYTF

SEQ ID NO: 102 - protein, 15 aa, Homo sapiens
CSARQGRDHTDTQYF

SEQ ID NO: 103 - protein, 15 aa, Homo sapiens
CSVAPAHTFTYEQYF

SEQ ID NO: 104 - protein, 11 aa, Homo sapiens
CSARGTTEAFF

SEQ ID NO: 105 - protein, 14 aa, Homo sapiens
CASRTTGTGEDYTF

SEQ ID NO: 106 - protein, 16 aa, Homo sapiens
CSAISGVLSPMNEQFF

SEQ ID NO: 107 - protein, 15 aa, Homo sapiens
CSARPPGGGGTEAFF

SEQ ID NO: 108 - protein, 12 aa, Homo sapiens
CSAGRGTDTQYF

SEQ ID NO: 109 - protein, 15 aa, Homo sapiens
CSAIRGSVNNSPLHF

SEQ ID NO: 110 - protein, 13 aa, Homo sapiens
CATMGPGTDTQYF

SEQ ID NO: 111 - protein, 15 aa, Homo sapiens
CASSLDWIGNQPQHF

SEQ ID NO: 112 - protein, 13 aa, Homo sapiens
CASSLDRGAPLHF

SEQ ID NO: 113 - protein, 13 aa, Homo sapiens
CSVVPAAVYGYTF

SEQ ID NO: 114 - protein, 14 aa, Homo sapiens
CASSQDRSGDTQYF

SEQ ID NO: 115 - protein, 12 aa, Homo sapiens
CSANPHTDTQYF

SEQ ID NO: 116 - protein, 12 aa, Homo sapiens
CASSTGGDGYTF

SEQ ID NO: 117 - protein, 13 aa, Homo sapiens
CASSDRMNTEAFF

SEQ ID NO: 118 - protein, 13 aa, Homo sapiens
CASTQGRNSPLHF

SEQ ID NO: 119 - protein, 12 aa, Homo sapiens
CSAPGQLEKLFF

SEQ ID NO: 120 - protein, 17 aa, Homo sapiens
CASSEGGRGDGNYGYTF

SEQ ID NO: 121 - protein, 15 aa, Homo sapiens
CAISVGAGQGETQYF

SEQ ID NO: 122 - protein, 13 aa, Homo sapiens
CASSLTGSRELFF

SEQ ID NO: 123 - protein, 14 aa, Homo sapiens
CSVWGDGSSYEQYF

SEQ ID NO: 124 - protein, 12 aa, Homo sapiens
CASSLDSSEQYF

SEQ ID NO: 125 - protein, 15 aa, Homo sapiens
CASSLLPSNTGELFF

SEQ ID NO: 126 - protein, 19 aa, Homo sapiens
CASSFRPGQGAGGANVLTF

SEQ ID NO: 127 - protein, 15 aa, Homo sapiens
CASTVYRGYNTEAFF

SEQ ID NO: 128 - protein, 15 aa, Homo sapiens
CASSLWISGLGEQFF

SEQ ID NO: 129 - protein, 15 aa, Homo sapiens
CASSSGTGQGETQYF

SEQ ID NO: 130 - protein, 13 aa, Homo sapiens
CASSDRTHDTQYF

SEQ ID NO: 131 - protein, 14 aa, Homo sapiens
CASSHRTENTEAFF

SEQ ID NO: 132 - protein, 16 aa, Homo sapiens
CASSYMGRASNTEAFF

SEQ ID NO: 133 - protein, 14 aa, Homo sapiens
CASNDRGLDGEQYF

SEQ ID NO: 134 - protein, 15 aa, Homo sapiens
CASSPRQGPTGELFF

SEQ ID NO: 135 - protein, 15 aa, Homo sapiens
CASSWTGTYQETQYF

SEQ ID NO: 136 - protein, 16 aa, Homo sapiens
CASSLQLAGGLETQYF

SEQ ID NO: 137 - protein, 14 aa, Homo sapiens
CSVEDLGGVDTQYF

SEQ ID NO: 138 - protein, 12 aa, Homo sapiens
CASSLEGNEQFF

SEQ ID NO: 139 - protein, 15 aa, Homo sapiens
CASRTKGGMNTEAFF

SEQ ID NO: 140 - protein, 20 aa, Homo sapiens
CASSRQGAYGAGGRYNEQFF

SEQ ID NO: 141 - protein, 12 aa, Homo sapiens
CSVDPGTGELFF

SEQ ID NO: 142 - protein, 13 aa, Homo sapiens
CSVGSGEGYEQYF

SEQ ID NO: 143 - protein, 11 aa, Homo sapiens
CSVDSDYEQYF

SEQ ID NO: 144 - protein, 15 aa, Homo sapiens
CASSDSGGVDYGYTF

SEQ ID NO: 145 - protein, 13 aa, Homo sapiens
CSATGANSYEQYF

SEQ ID NO: 146 - protein, 12 aa, Homo sapiens
CSVGGSFYGYTF

SEQ ID NO: 147 - protein, 16 aa, Homo sapiens
CASSDPAGVFRQPQHF

SEQ ID NO: 148 - protein, 17 aa, Homo sapiens
CASSLETTSSNTGELFF

SEQ ID NO: 149 - protein, 13 aa, Homo sapiens
CASSQDPPDTQYF

SEQ ID NO: 150 - protein, 15 aa, Homo sapiens
CASSSPGGANYEQFF

SEQ ID NO: 151 - protein, 15 aa, Homo sapiens
CASSPPPGVKETQYF

SEQ ID NO: 152 - protein, 16 aa, Homo sapiens
CSARSLRTVHNEKLFF

SEQ ID NO: 153 - protein, 18 aa, Homo sapiens
CASIWDRTPPTGVNGYTF

SEQ ID NO: 154 - protein, 13 aa, Homo sapiens
CASSSYGQETQYF

SEQ ID NO: 155 - protein, 15 aa, Homo sapiens
CASSEWTAPNQPQHF

SEQ ID NO: 156 - protein, 14 aa, Homo sapiens
CASSPTSNLGTQYF

SEQ ID NO: 157 - protein, 12 aa, Homo sapiens
CSVVAGIYGYTF

SEQ ID NO: 158 - protein, 13 aa, Homo sapiens
CASTRGDGTEAFF

SEQ ID NO: 159 - protein, 15 aa, Homo sapiens
CASSQETALNSPLHF

SEQ ID NO: 160 - protein, 16 aa, Homo sapiens
CASSLVKRGAYNEQFF

SEQ ID NO: 161 - protein, 13 aa, Homo sapiens
CASSSVGGDTQYF

SEQ ID NO: 162 - protein, 14 aa, Homo sapiens
CASSIPRQGSGYTF

SEQ ID NO: 163 - protein, 14 aa, Homo sapiens
CASSIEGQKNIQYF

SEQ ID NO: 164 - protein, 15 aa, Homo sapiens
CASSPSAVGTDTQYF

SEQ ID NO: 165 - protein, 15 aa, Homo sapiens
CASSPVDPMGQPQHF

SEQ ID NO: 166 - protein, 15 aa, Homo sapiens
CASSITSGSYNEQFF

SEQ ID NO: 167 - protein, 13 aa, Homo sapiens
CASSTTGNTEAFF

SEQ ID NO: 168 - protein, 18 aa, Homo sapiens
CAGRGTGQRMGYNSPLHF

SEQ ID NO: 169 - protein, 16 aa, Homo sapiens
CASSLEATGAYNEQFF

SEQ ID NO: 170 - protein, 15 aa, Homo sapiens
CASSFGGGNTDTQYF

SEQ ID NO: 171 - protein, 13 aa, Homo sapiens
CASSYWAAGELFF

SEQ ID NO: 172 - protein, 17 aa, Homo sapiens
CASSQSMGRTNTGELFF

SEQ ID NO: 173 - protein, 13 aa, Homo sapiens
CASSYLAGDIQYF

SEQ ID NO: 174 - protein, 14 aa, Homo sapiens
CASSYFSSSYEQYF

SEQ ID NO: 175 - protein, 14 aa, Homo sapiens
CASSLQALNTEAFF

SEQ ID NO: 176 - protein, 14 aa, Homo sapiens
CASSLAEDSYEQYF

SEQ ID NO: 177 - protein, 15 aa, Homo sapiens
CASSDSGGSNNEQFF

SEQ ID NO: 178 - protein, 14 aa, Homo sapiens
CASRKQGGNYGYTF

SEQ ID NO: 179 - protein, 19 aa, Homo sapiens
CASSRQPGQGAGGANVLTF

SEQ ID NO: 180 - protein, 16 aa, Homo sapiens
CASSYSPGLAGDEQYF

SEQ ID NO: 181 - protein, 15 aa, Homo sapiens
CAISESGRSTDTQYF

SEQ ID NO: 182 - protein, 13 aa, Homo sapiens
CASSLTYSYEQYF

SEQ ID NO: 183 - protein, 15 aa, Homo sapiens
CASRGEQGFRQPQHF

SEQ ID NO: 184 - protein, 11 aa, Homo sapiens
CSVGTSGTQYF

SEQ ID NO: 185 - protein, 13 aa, Homo sapiens
CASSRLQGNTQYF

SEQ ID NO: 186 - protein, 13 aa, Homo sapiens
CASSFREKYEQYF

SEQ ID NO: 187 - protein, 14 aa, Homo sapiens
CASTLAGGHEKLFF

SEQ ID NO: 188 - protein, 15 aa, Homo sapiens
CAIPAGSMDTGELFF

SEQ ID NO: 189 - protein, 17 aa, Homo sapiens
CASSLLGGTLSTGELFF

SEQ ID NO: 190 - protein, 19 aa, Homo sapiens
CASSFFFIPRTGGNYGYTF

SEQ ID NO: 191 - protein, 15 aa, Homo sapiens
CASSLGTGWAGELFF

SEQ ID NO: 192 - protein, 14 aa, Homo sapiens
CASSPDRVRETQYF

SEQ ID NO: 193 - protein, 16 aa, Homo sapiens
CASSFWGAGGREKLFF

SEQ ID NO: 194 - protein, 15 aa, Homo sapiens
CASSGISGNPYEQYF

SEQ ID NO: 195 - protein, 12 aa, Homo sapiens
CASSLGDTEAFF

SEQ ID NO: 196 - protein, 15 aa, Homo sapiens
CASSARDTFTGELFF

SEQ ID NO: 197 - protein, 12 aa, Homo sapiens
CASSPLPSEQYF

SEQ ID NO: 198 - protein, 13 aa, Homo sapiens
CASSPWGSNEQFF

SEQ ID NO: 199 - protein, 14 aa, Homo sapiens
CASGRTGSYNEQFF

SEQ ID NO: 200 - protein, 14 aa, Homo sapiens
CAWSKLGPPDTQYF

SEQ ID NO: 201 - protein, 18 aa, Homo sapiens
CSARDQTSGSSYPETQYF

SEQ ID NO: 202 - protein, 13 aa, Homo sapiens
CASSYLGRDTQYF

SEQ ID NO: 203 - protein, 12 aa, Homo sapiens
CASSLGDTEAFF

SEQ ID NO: 204 - protein, 14 aa, Homo sapiens
CASGPPGGADGYTF

SEQ ID NO: 205 - protein, 15 aa, Homo sapiens
CASSLVRGRAGELFF

SEQ ID NO: 206 - protein, 14 aa, Homo sapiens
CASSPTGLSRGAFF

SEQ ID NO: 207 - protein, 16 aa, Homo sapiens
CASSKNTGIRTDTQYF

SEQ ID NO: 208 - protein, 14 aa, Homo sapiens
CARSGTGIVNEQFF

SEQ ID NO: 209 - protein, 12 aa, Homo sapiens
CASSQDRREAFF

SEQ ID NO: 210 - protein, 19 aa, Homo sapiens
CASSHQILPGTGPGNTIYF

SEQ ID NO: 211 - protein, 12 aa, Homo sapiens
CSARDGTGNGYT

SEQ ID NO: 212 - protein, 12 aa, Homo sapiens
CSARDRTGNGYT

SEQ ID NO: 213 - protein, 12 aa, Homo sapiens
CSARDRVGNTIY

SEQ ID NO: 214 - protein, 12 aa, Homo sapiens
CSARVGVGNTIY

SEQ ID NO: 215 - protein, 13 aa, Homo sapiens
CSVGTGGTNEKLF

SEQ ID NO: 216 - protein, 11 aa, Homo sapiens
CSSQEGGYGYT

SEQ ID NO: 217 - protein, 12 aa, Homo sapiens
CSARDQTGNGYT

SEQ ID NO: 218 - protein, 12 aa, Homo sapiens
CSARDRIGNGYT

SEQ ID NO: 219 - protein, 12 aa, Homo sapiens
CSARIGVGNTIY

SEQ ID NO: 220 - protein, 12 aa, Homo sapiens
CSARSGVGNTIY

SEQ ID NO: 221 - protein, 14 aa, Homo sapiens
CASSEGRVSPGELF

SEQ ID NO: 222 - protein, 13 aa, Homo sapiens
CSVGQGGTNEKLF

SEQ ID NO: 223 - protein, 13 aa, Homo sapiens
CASSLGQAYEQYF

SEQ ID NO: 224 - protein, 14 aa, Homo sapiens
CAISTGDSNQPQHF

SEQ ID NO: 225 - protein, 14 aa, Homo sapiens
CAIGTGDSNQPQHF

SEQ ID NO: 226 - protein, 14 aa, Homo sapiens
CATSTGDSNQPQHF

SEQ ID NO: 227 - protein, 12 aa, Homo sapiens
CASSARSGELFF

SEQ ID NO: 228 - protein, 12 aa, Homo sapiens
CASSARTGELFF

SEQ ID NO: 229 - protein, 12 aa, Homo sapiens
CASSVRTGELFF

SEQ ID NO: 230 - protein, 15 aa, Homo sapiens
CASRYRDDSYNEQFF

SEQ ID NO: 231 - protein, 13 aa, Homo sapiens
CASSSRSSYEQYF

SEQ ID NO: 232 - protein, 13 aa, Homo sapiens
CASSIRSSYEQYF

SEQ ID NO: 233 - protein, 13 aa, Homo sapiens
CASSMRSSYEQYF

SEQ ID NO: 234 - protein, 13 aa, Homo sapiens
CASSTRSTDTQYF

SEQ ID NO: 235 - protein, 13 aa, Homo sapiens
CASSMRSTDTQYF

SEQ ID NO: 236 - protein, 13 aa, Homo sapiens
CASSIRSAYEQYF

SEQ ID NO: 237 - protein, 12 aa, Homo sapiens
CASSTGSYGYTF

SEQ ID NO: 238 - protein, 12 aa, Homo sapiens
CASSIGNYGYTF

SEQ ID NO: 239 - protein, 11 aa, Homo sapiens
CASSSANYGYT

SEQ ID NO: 240 - protein, 15 aa, Homo sapiens
CASSLAPGATNEKLF

SEQ ID NO: 241 - protein, 11 aa, Homo sapiens
CASSSAHYGYT

SEQ ID NO: 242 - protein, 12 aa, Homo sapiens
CASSLEGYTEAF

SEQ ID NO: 243 - protein, 11 aa, Homo sapiens
CASSSAYYGYT

SEQ ID NO: 244 - protein, 13 aa, Homo sapiens
CASSLVGGRYGYT

SEQ ID NO: 245 - protein, 10 aa, Homo sapiens
CASSVVNEQF

SEQ ID NO: 246 - protein, 15 aa, Homo sapiens
CASSLTSGSPYNEQF

SEQ ID NO: 247 - protein, 12 aa, Homo sapiens
CASSFQGYTEAF

SEQ ID NO: 248 - protein, 14 aa, Homo sapiens
CASRPGTSSYEQYF

SEQ ID NO: 249 - protein, 17 aa, Homo sapiens
CASSPQTGTGGYGYTFG

SEQ ID NO: 250 - protein, 13 aa, Homo sapiens
CASSVWGTDTQYF

SEQ ID NO: 251 - protein, 14 aa, Homo sapiens
CASRRQGRDNEQFF

SEQ ID NO: 252 - protein, 14 aa, Homo sapiens
CATRIGWGTDTQYF

SEQ ID NO: 253 - protein, 9 aa, Homo sapiens
GLCTLVAML

SEQ ID NO: 254 - protein, 14 aa, Homo sapiens
CSVGSGGTNEKLFF